(19) United States
(12) Patent Application Publication
(10) Pub. No.: US 2013/0190833 A1
(43) Pub. Date: Jul. 25, 2013

(54) Use of Electric Fields for Reducing Patient Discomfort During Defibrillation

(71) Applicants: Lazaro Salomon Azar, Givatayim (IL); Avi Allon Livnat, Tel Aviv (IL); Yaron Hefetz, Kibbutz Alonim (IL)

(72) Inventors: Lazaro Salomon Azar, Givatayim (IL); Avi Allon Livnat, Tel Aviv (IL); Yaron Hefetz, Kibbutz Alonim (IL)

(73) Assignee: SMARTWAVE MEDICAL LTD., Tel-Aviv (IL)

(21) Appl. No.: 13/811,863

(22) PCT Filed: Jul. 21, 2011
(86) PCT No.: PCT/US11/44771

§ 371 Date: Apr. 4, 2013

Related U.S. Application Data
(60) Provisional application No. 61/400,128, filed on Jul. 23, 2010.

(51) Int. Cl. A61N 1/39

(52) U.S. Cl. CPC A61N 1/3918
USPC 607/5

(57) ABSTRACT

Devices, systems and methods for reducing patent discomfort during defibrillation by delivering pulses to electrode configurations that create electric fields confined to and/or concentrated in an area of fibrillation are described. Embodiments provide for an implantable defibrillator having an electrode lead system having at least one electrode lead and at least one three electrodes, a controller for determining whether fibrillation exists and a voltage generator for discharging one or more defibrillation pulses to the at least three electrodes to create electric fields having different directions and high electric field concentrations in areas of the heart needing defibrillation and low electric field concentrations outside those areas.

### CROSS-REFERENCE TO RELATED APPLICATIONS

The present application claims priority to U.S. Provisional Patent Application No. 61/400,128, filed on Jul. 23, 2010 and entitled “Implantable Atrial Defibrillator and Defibrillation Methods,” the disclosure of which is incorporated herein by reference in its entirety.

### FIELD

Devices, systems and methods relating to atrial defibrillation are described herein. Embodiments of the present disclosure more specifically reduce patient discomfort during defibrillation by delivering pulses to electrodes positioned in or around the heart that create electric fields confined to and/or concentrated in an area of fibrillation. In particular, the disclosed subject matter minimizes the magnitude of electric fields outside areas needing defibrillation by configuring the electric fields to intersect each other at different angles and/or using one or more electrodes to confine those fields to target defibrillation zones.

### BACKGROUND

Atrial Fibrillation (“AF”) is the most common cardiac arrhythmia involving at least one of the left or right atrium of the heart. One way to defibrillate an atrium is by delivering electrical defibrillation pulses to the heart at specific times during the cardiac cycle. Systems and devices for delivering these pulses may be external to and/or implanted within the body. Atrial defibrillation using an implantable atrial defibrillator generally includes automatically detecting AF and automatically delivering one or more electrical pulses to the left and/or right atrium of the heart. Delivering an electrical pulse may be intolerably painful for a patient and discourage the use of automatic implantable atrial defibrillators, particularly when the energy delivered is too high. Conversely, delivering an electrical pulse having an energy that is too low will result in an unsuccessful defibrillation attempt. Atrial defibrillation should therefore be tolerable and effective and/or reduce patient discomfort.

Various pulse and electrode configurations directed to more efficient and less painful atrial defibrillation techniques are known. International Patent Application No. PCT/US2009/033786, filed on Feb. 11, 2009, entitled “Atrial Defibrillation Using an Implantable Defibrillation System” and incorporated herein by reference, discloses an implantable atrial defibrillator having an electrode lead system that is configured for delivering short pulses of high voltage to minimize patient discomfort.

“What is the Optimal Electrode Configuration for Atrial Defibrillators in Man?,” authored by A. R. J. Mitchell et al. and published by The European Society of Cardiology, discloses experimental results showing that right atrium to coronary sinus electrode configurations may significantly reduce the atrial defibrillation threshold, i.e., the voltage required to effectively defibrillate an atrium.

U.S. Pat. No. 5,813,999 to Ayers et al., filed on Dec. 21, 1995 and entitled “Implantable Atrial Defibrillator Providing Reduced Cardioversion Discomfort,” discloses an implantable atrial cardioverter that seeks to reduce patient discomfort by delivering cardioverting energy to the atria of a heart having a biphasic waveform with a first phase and a second phase of opposite polarity and equal time duration.

U.S. Pat. No. 7,596,409 to Berger, filed Oct. 9, 2003 and entitled “Cardiac Shock Electrode System and Corresponding Implantable Defibrillator System,” discloses an implantable cardioverter defibrillator system having an internal electrode placed in the right ventricle of the heart and a quasi-Faraday cage with one or more electrodes placed over a significant portion of the heart. This patent discloses that defibrillation shocks can be applied between the internal electrode in the ventricle and the electrode(s) of the quasi-Faraday cage and indicates that the quasi-Faraday cage is capable of confining a significant portion of the electric fields to the heart to reduce the pain felt by a patient.

U.S. Pat. No. 5,279,291 to Adams et al., filed on Aug. 3, 1992 and entitled “Method for Atrial Defibrillation,” discloses an implantable atrial defibrillator that provides a defibrillation pulse to the atria of the heart synchronized with sensed R-waves in response to non-coincident sensing of an R-wave at first and second areas of the heart.

U.S. Pat. No. 6,327,500 to Cooper et al., filed on Nov. 15, 2009 and entitled “Dual Shock Atrial Defibrillation Apparatus,” discloses an implantable system for defibrillating the atria of a heart having a pair of electrodes configured for delivering a first pulse in the heart and a pulse generator operatively associated with the first pair of electrodes for delivering the first pulse. The patent discloses that the pulse generator may deliver a second pulse after the first pulse without intervening monitoring to reduce the voltage necessary for the pulse and thus the pain associated therewith.

U.S. Patent Application Publication No. 2004/0093054 to Zheng et al., filed on Nov. 3, 2003 and entitled “Inter-Atrial Septum Electrode for Atrial Defibrillation,” discloses an implantable atrial defibrillation system including (i) a first catheter insertable into the right atrium of the heart and having a first electrode positioned at the atrial septum of the heart as an atrial septum electrode, (ii) a second electrode which together with the first electrode forms an electrode pair and (iii) a pulse generator operatively associated with the electrode pair for delivering a first pulse. The application discloses that the second electrode may be positioned through the coronary sinus ostium and in the coronary sinus or a vein on the surface of the left ventricle, such as the great vein. An additional electrode may be positioned in the superior vena cava, right atrium (including the right atrial appendage) or the right ventricle may also be included and the pulse generator may concurrently deliver a first pulse through the additional electrode and the atrial septum electrode and a second pulse through the atrial septum electrode and the second electrode.

International Patent Application No. PCT/US2011/029509, filed on Mar. 23, 2011, entitled “Modes of Operation for Atrial Defibrillation Using an Implantable Defibrillator” and incorporated herein by reference, discloses modes of operation for defibrillating the atria using an implantable defibrillator and remote modes of operation enabling wireless communication between an implantable atrial defibrillator and a server.

International Patent Application No. PCT/US2011/041411, filed on Jun. 22, 2011, entitled “Pulse Parameters and Electrode Configurations for Reducing Patient Discomfort from Defibrillation” and incorporated herein by reference, discloses defibrillating an atrium with one or more high-voltage, short-duration pulses using one or more pairs of electrodes positioned in or around the heart.

### SUMMARY

Some embodiments described herein are directed to an implantable defibrillator having an electrode lead system with at least one electrode lead and at least three electrodes, a controller configured to determine whether a heart is fibrillating and emit a command signal if fibrillation exists and a voltage generator in communication with the electrode lead system and the controller to sequentially discharge at least a first defibrillation pulse and second defibrillation pulse to the electrode lead system after receiving the command signal. In some embodiments, the first defibrillation pulse may create a first electric field across two of the at least three electrodes and the second defibrillation pulse may create a second electric field across two of the at least three electrodes in a direction different from that of the first electric field. The electrode lead system may include a first electrode, a second electrode, a third electrode and a fourth electrode positioned along a single electrode lead, according to some embodiments, wherein the first electrode and the second electrode form a first electrode pair and the third electrode and the fourth electrode form a second electrode pair. Further embodiments may include the first electrode being positioned in the right ventricle, the second electrode and third electrode being positioned in the pulmonary artery and the fourth electrode being positioned in the right atrium. In other embodiments, the first electrode may be positioned in the right atrium, the second electrode and third electrode may be positioned in the pulmonary artery and the fourth electrode may be positioned in the right ventricle.

In some embodiments, the electrode lead system may include a first electrode, a second electrode and a third electrode positioned along a single electrode lead, wherein the first electrode and the second electrode form a first electrode pair and the third electrode and one of the first electrode or the second electrode form a second electrode pair. Further embodiments may include the first electrode being positioned in the right atrium, the second electrode being positioned in the right ventricle and the third electrode being positioned in the pulmonary artery. In other embodiments, the first electrode may be positioned in the right atrium, the second electrode may be positioned in the pulmonary artery and the third electrode may be positioned in the right ventricle. In other embodiments, the first electrode may be positioned in the pulmonary artery, the second electrode may be positioned in the right atrium and the third electrode may be positioned in the right ventricle. In other embodiments, the first electrode may be positioned in the right ventricle, the second electrode may be positioned in the right atrium and the third electrode may be positioned in the pulmonary artery.

Some embodiments of the present disclosure may be directed to an electrode lead system having a first electrode, a second electrode, a third electrode and a fourth electrode positioned along a single electrode lead, wherein the first electrode and the fourth electrode receive a negative voltage from the voltage generator and the second electrode and the third electrode receive a positive voltage from the voltage generator to form the first electric field between the first electrode and the second electrode and the first electric field between the third electrode and the fourth electrode. Some embodiments may also include the first electrode and the second electrode receiving a positive voltage from the voltage generator and the third electrode and the fourth electrode receiving a negative voltage from the voltage generator to form the second electric field between the first electrode and the fourth electrode and the second electric field between the second electrode and the third electrode. In some embodiments, the first electric fields and the second electric fields may intersect each other at different angles to form a high concentration of electrical pulses within a defibrillation zone of the heart.

Some implantable defibrillator embodiments disclosed herein may implement at least one of the at least three electrodes as a confinement electrode configured to control the distribution of at least one of the first electric field or the second electric field. In some embodiments, at least one of the at least three electrodes may be an extended electrode, at least one of the at least three electrodes may be positioned in a ventricle for ventricular defibrillation and/or the electrode lead system further comprises two single leads.

Some method embodiments of the present disclosure are directed to defibrillating the heart with an implantable defibrillator by positioning an electrode lead system having at least three electrodes in, on and/or around the heart, determining whether the heart is fibrillating and sending a command signal to a voltage generator if the heart is fibrillating. Furthermore, embodiments may involve generating at the voltage generator at least a first defibrillation pulse and second defibrillation pulse and delivering the first defibrillation pulse and the second defibrillation pulse to the electrode lead system. In some embodiments, the first defibrillation pulse may create a first electric field across two of the at least three electrodes and the second defibrillation pulse may create a second electric field across two of the at least three electrodes in a direction different from that of the first electric field.

In some method embodiments, the electrode lead system includes a first electrode, a second electrode, a third electrode and a fourth electrode positioned along a single electrode lead, wherein the first electrode and the second electrode may form a first electrode pair and the third electrode and the fourth electrode may form a second electrode pair. In some embodiments, the electrode lead system may include a first electrode, a second electrode and a third electrode positioned along a single electrode lead, wherein the first electrode and the second electrode may form a first electrode pair and the third electrode and one of the first electrode or the second electrode may form a second electrode pair. According to some embodiments, the electrode lead system may have a first electrode, a second electrode, a third electrode and a fourth electrode positioned along a single electrode lead, wherein the first electrode and the fourth electrode receive a negative voltage from the voltage generator and the second electrode and the third electrode receive a positive voltage from the voltage generator to form the first electric field between the first electrode and the second electrode and the first electric field between the third electrode and the fourth electrode. Some embodiments may also include the first electrode and the second electrode receiving a positive voltage from the voltage generator and the third electrode and the fourth electrode receiving a negative voltage from the voltage generator to form the second electric field between the first electrode and the fourth electrode and the second electric field between the second electrode and the third electrode.

Some method embodiments may involve an implantable defibrillator having at least one electrode as a confinement electrode configured to control the distribution of at least one of the first electric field or the second electric field. In some embodiments, at least one electrode may be an extended electrode and/or positioned in a ventricle for ventricular defibrillation. In some embodiments, the electrode lead system may include two single leads.

Some device embodiments of the present disclosure may also be directed to an implantable defibrillator that includes an electrode lead system having at least one electrode lead and at least three electrodes, a controller configured to determine whether a heart is fibrillating and emit a command signal if fibrillation exists and/or a voltage generator in communication with the electrode lead system and the controller to sequentially discharge at least one defibrillation pulse to the electrode lead system after receiving the command signal. Some embodiments may further include at least one of the at least three electrodes as an electric field confinement electrode configured to confine one or more electric fields created by the at least one defibrillation pulse. In some embodiments, the electrode lead system may have a first electrode and a second electrode positioned along a single electrode lead and forming a first electrode pair and at least one electric field confinement electrode. In some embodiments, the first electric field may be formed between the first electrode and the second electrode and substantially confined to a defibrillation zone of the heart by the at least one electric field confinement electrode. In further embodiments, the first electrode may have a positive voltage, the second electrode may have a negative voltage and one or more of the at least one electric field confinement electrode may have a voltage potential value between the positive voltage of the first electrode and the negative voltage of the second electrode. The electrode lead system may also include two single electrode leads, wherein at least one electric field confinement electrode is position on each of the two single electrode leads.

The subject matter of the present disclosure may also be directed to heart defibrillation systems having a defibrillator configured to be implanted in a patient. In some embodiments, the defibrillator may include an electrode lead system having at least one electrode lead and at least three electrodes, a controller configured to determine whether a heart is fibrillating and emit a command signal if fibrillation exists and/or a voltage generator in communication with the electrode lead system and the controller to sequentially discharge at least a first defibrillation pulse and second defibrillation pulse to the electrode lead system after receiving the command signal. In some embodiments, the system may further involve the first defibrillation pulse creating a first electric field across two of the at least three electrodes and the second defibrillation pulse creating a second electric field across two of the at least three electrodes in a direction different from that of the at least one first electric field.

Other system embodiments may be directed to heart defibrillation systems having a defibrillator configured to be implanted in a patient, where the defibrillator includes an electrode lead system having at least one electrode lead and at least three electrodes, a controller configured to determine whether a heart is fibrillating and emit a command signal if fibrillation exists and/or a voltage generator in communication with the electrode lead system and the controller to sequentially discharge at least a first defibrillation pulse to the electrode lead system after receiving the command signal. In some embodiments, at least one of the at least three electrodes may be an electric field confinement electrode configured to confine one or more electric fields created by the at least one first defibrillation pulse. System embodiments may also include a communication device disposed outside the patient and configured to communicate with the defibrillator.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE FIGURES

FIG. 1 shows a block diagram of an implantable atrial defibrillator according to some embodiments of the present disclosure.

FIG. 2 shows a defibrillation system according to some embodiments of the present disclosure.

FIGS. 3A and 3B show defibrillation pulse train waveforms produced according to some embodiments of an implantable defibrillator of the present disclosure.

FIG. 4 graphically depicts why defibrillation efficiency may be increased by using more than one electrode pair to deliver defibrillation pulses according to some embodiments of the present disclosure.

FIGS. 5A-5C show configurations of three of more electrodes for producing electric fields in different directions according to some embodiments of the present disclosure.

FIG. 5D shows two extended electrodes for producing a uniform electric field according to some embodiments of the present disclosure.

FIG. 6A shows an electric field created between a positively-charged electrode and a negatively-charged electrode as known in the art.

FIGS. 6B and 6C show the effect of one or more confinement electrodes on the electric field produced between a positively-charged electrode and a negatively-charged electrode according to some embodiments of the present disclosure.

FIGS. 7A-7D show configurations of two or more electrodes located along one or more leads for defibrillating the heart according to some embodiments of the present disclosure.

FIG. 8 shows a known configuration of five electrodes positioned along two leads for defibrillating the heart.

FIGS. 9A-9D show configurations of three or more electrodes positioned in series along a single lead for atrial defibrillation according to some embodiments of the present disclosure.

FIG. 9E shows a configuration of five electrodes positioned in series along a first single lead and one electrode positioned on a second single lead for atrial defibrillation according to some embodiments of the present disclosure.

FIG. 10 shows a configuration of five electrodes positioned within the heart for atrial defibrillation according to some embodiments of the present disclosure.

FIG. 11 shows a configuration of five electrodes positioned in series along a first single lead, one electrode positioned on a second single lead and one electrode positioned on a branch lead of the first single lead and extending into the right ventricle for enabling ventricular defibrillation according to some embodiments of the present disclosure.

Like reference symbols in the various drawings indicate like elements.

### DETAILED DESCRIPTION

The subject matter described herein relates to defibrillating the left and/or right atrium of the heart using an implantable atrial defibrillation system and is not limited in its application to the details set forth in the following disclosure or exemplified by the illustrative embodiments. The subject matter is capable of other embodiments and of being practiced or carried out in various ways. Features of the present disclosure, which are, for clarity, described in the context of separate embodiments, may also be provided in combination in a single embodiment. Conversely, various features of the present disclosure, which are, for brevity, described in the context of a single embodiment, may also be provided separately or in any suitable sub-combination or any other described embodiment of the present disclosure. Certain features described in the context of various embodiments are not to be considered essential features of those embodiments, unless the embodiment is inoperative without those elements.

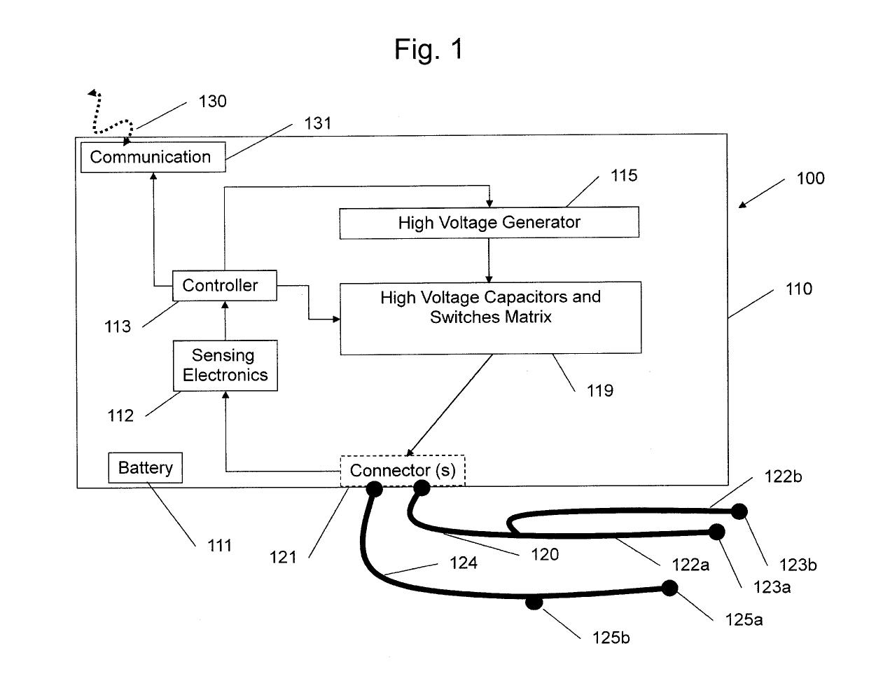

FIG. 1 shows a block diagram of an implantable atrial defibrillator (“IAD”) (100) according to some embodiments of the present disclosure. The internal construction of the IAD (100) may vary depending upon the embodiment and, in some embodiments, may be an internal construction that is known in the art. Example configurations of the IAD (100) are provided in International Patent Application No. PCT/US2009/033786, filed on Feb. 11, 2009, entitled “Atrial Defibrillation Using an Implantable Defibrillation System,” the disclosure of which is incorporated herein by reference in its entirety.

For performing the defibrillation methods contemplated by the present disclosure, the IAD (100) may include a communication transceiver (131) capable of wirelessly communicating with an external device using a communication link (130). The communication link (130) may have short-range and/or long-range capabilities. The communication link (130) may be an ultrasonic link communicating with an external device in contact with a patient's body. In some embodiments, the communication link (130) may be a short-range radio frequency (“RF”) communication link and may use a proprietary protocol for communicating with an interface device. In some embodiments, the communication link (130) may use a common protocol, such as Bluetooth technology or wireless fidelity (“Wi-Fi”), wherein the external device may include mobile devices (i.e., portable devices), such as, for example, a mobile phone, media player, smart phone, Personal Digital Assistant (“PDA”) and other handheld computing devices and the like.

The IAD (100) may have a main body (110). The main body (110) may be made of one or more bio-compatible materials known in the art. The main body (110) may contain at least one battery (111) and electronic circuitry for sensing cardiac activity, processing the sensed activity to determine whether the activity is normal or indicative of a fibrillation state, and delivering one or more high-voltage defibrillation pulses. In some embodiments, the IAD (100), and in particular the electronic circuitry may be configured to differentiate between atrial and ventricular fibrillations and respond accordingly based on whether the atria or ventricles of the heart are fibrillating.

Some embodiments of the main body (110) may include at least one electrical connector (121) connected to one or more leads. In some embodiments, at least one of the one or more leads may be permanently attached to the main body (110). As shown in FIG. 1, some embodiments may include a first lead (120) and/or a second lead (124). In some embodiments, the lead (120) may be bifurcated into sub-leads (122a) and (122b) having exposed electrodes (123a) and (123b), respectively. In some embodiments, the second lead (124) may be a single lead having a distal electrode (125a) and a central electrode (125b) at some distance from the distal electrode (125a). The number and configuration of leads, sub-leads and electrodes will vary depending on the embodiment. For example, some embodiments of the IAD (100) may position one or more electrodes in the left and/or right atrium for pacing the heart, in addition to those electrodes used for atrial defibrillation. In some embodiments, one or more additional electrodes may be positioned in the right ventricle and used for electrocardiogram (“ECG”) sensing and delivering one or more ventricular defibrillation pulses or pulse trains. In some embodiments, the main body (110), or parts thereof, may be used as an electrode. In some embodiments, the communication transceiver (131) may use the lead (120) as an antenna for RF communication. Some embodiments of the IAD (100) may include a dedicated antenna, for example a coil, loop or dipole antenna, located within or outside the main body (110).

At least one of the electrodes (e.g., 123a, 123b, 125a and/or 125b) may be used for sensing ECG signals for monitoring the cardiac activity of a patient implanted with the IAD (100). In some embodiments, one or more electrodes may be used for both sensing ECG data and delivering defibrillation pulses or cardiac pacing. In some embodiments, at least one electrode may be dedicated to sensing ECG signals. Embodiments of the IAD (100) may include a sensing electronic module (112) configured to condition (e.g., amplify and/or filter) the ECG signals. The IAD (100) may include additional sensors for monitoring cardiac activity and other bodily functions. For example, the IAD (100) may include one or more thermal sensors to monitor patient body temperature, blood oxygenation sensors, microphones to monitor sound emitted from the heart and the respiratory system, breathing sensors (e.g., capacitive sensors or sensors sensing the bending of a lead or sub-lead due to breathing) and/or other sensors known in the art. In some embodiments, sensor electronics may include an Analog-to-Digital Converter (“ADC”).

The IAD (100) may include a controller (113) for performing signal conditioning and analysis. The controller (113) may receive data indicative of cardiac activity from the sensing electronics (112) and other optional sensors and may receive commands and data from the communication transceiver (131). The controller (113) may determine the state of the cardiac activity based on ECG signals and other sensor data and control the pulse-generating circuitry to produce one or more defibrillation pulses when appropriate. In some embodiments, pulse-generating circuitry may include a high-voltage generator (115) and a high-voltage capacitor and switches matrix (119) configured to produce high-voltage, short-duration pulses for defibrillating the atria and/or ventricles of the heart. In some embodiments, atrial defibrillation may be done using low-energy (e.g., <2 J), high-voltage (e.g., >600 V), short-duration (e.g., <500 μs) pulses. Other exemplary energy, voltage and/or pulse duration ranges are set forth in International Patent Application No. PCT/US2011/041411, filed on Jun. 22, 2011, entitled “Pulse Parameters and Electrode Configurations for Reducing Patient Discomfort from Defibrillation” and incorporated herein by reference in its entirety. In other embodiments, a train of two or more pulses may be used. In some embodiments, the IAD (100) may be configured as an atrial defibrillator and pacemaker, an atrial defibrillator and ventricular defibrillator (also known as an implantable cardioverter-defibrillator, or “ICD”) or an atrial defibrillator, ventricular defibrillator and pacemaker. The IAD (100) may be able to monitor, detect and collect data relating to cardiac activity, analyze whether a cardiac condition exists and deliver a defibrillation and/or pacing therapy that best treats the condition. Analyzing the cardiac activity and identifying the existence of a condition may be performed by the controller (113) of the IAD (100), in conjunction with other circuitry and software within the IAD (100). Alternatively, or in addition, cardiac activity analyses and processing may be performed remotely by a medical facility that receives the collected data over the communication link (130).

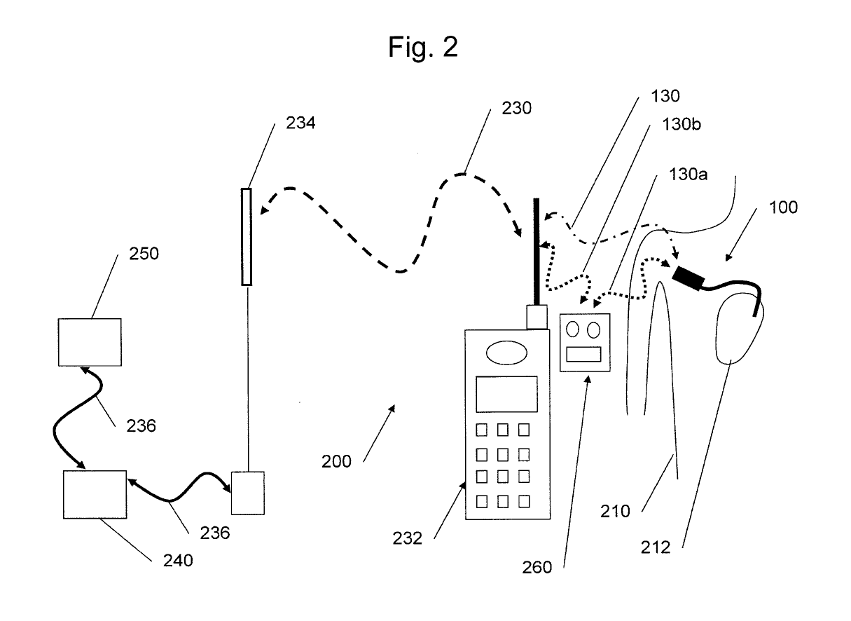

FIG. 2 shows a defibrillation system (200) using an embodiment of the IAD (100) according to the subject matter of the present disclosure. In some embodiments of the system (200), the IAD (100) may be implanted in a patient (210). One or more electrodes (122) may be positioned in or around the atria of the heart (212) of the patient (210). The system (200) also includes an external communication device (232), an interface device (260) and a server (240), all of which may be in wireless communication with one another. In some embodiments, the IAD (100) may communicate directly with the server (240) or via the external communication device (232) and/or the interface device (260) to, for example, transmit data to the server (240) relating to a possible AF state.

The IAD (100) of the system (200) may communicate with the external communication device (232). The communication between the IAD (100) and the external communication device (232) may be short-range and/or long-range communication. The external communication device (232) may be configured as a two-way communicator capable of transmitting and receiving both data and voice information or, alternatively, the external communication device (232) may be configured to transmit and receive only data or only voice information. In some embodiments, the external communication device (232) may include one or more user inputs, such as a keypad, touch screen, scroll wheel or microphone. Some embodiments of the external communication device (232) may have one or more user outputs, such as a display screen, speaker, vibrating mechanism and/or light-emitting component (e.g., a light-emitting diode). The external communication device (232) may also include a global positioning system (“GPS”) receiver for determining the location of the external communication device (232). The external communication device (232) may be a cellular phone, a smartphone or any other handheld computing device. In some embodiments, external communication device (232) may also be a satellite communication device.

In some embodiments, the IAD (100) may communicate with the external communication device (232) via the communication link (130), as shown in FIG. 2. The IAD (100) may, in some embodiments, communicate with the external communication device (232) via an interface device (260). In some embodiments, the interface device (260) may be an application embedded within external communication device (232). In some embodiments, the external communication device (232) and/or the interface device (260) may be embedded within the IAD (100) itself, either as software and/or hardware components of the IAD (100). Other embodiments of the present disclosure contemplate the interface device (260) as a separate component in wireless communication with IAD (100), server (240) and/or external communication device (232). In such embodiments, the interface device (260) may be any shape or size. The interface device (260) may be miniature for discreet placement in or around the heart (212) of the patient (210). The interface device (260), in some embodiments, may be used primarily for providing an interface between the IAD (100) and the external communication device (232) and, thus, may contain no user inputs or outputs. In other embodiments, the interface device (260) may communicate directly with the server (240). The interface device (260) may include user inputs, such as switches or buttons, and user outputs, such as a display screen, speaker(s) and/or vibrating mechanism. In some embodiments, interface device (260) or external communication device (232) may be used by to control the operation of the IAD (100). In some embodiments, server (240) may use interface device (260) or external communication device (232) to remotely control the operation of IAD (100). Communication between the IAD (100) and the external communication device (232) via the interface device (260) may involve using short-range channels. As shown in FIG. 2, the IAD (100) may communicate with the interface device (260) via a short-range channel (130a) and the interface device (260) may communicate with the external communication device (232) via a short-range channel (130b). In some embodiments, the channels connecting the IAD (100), interface device (260) and external communication device (232) may be long-range channels or a combination of short-range and long-range channels.

FIG. 2 also shows that the external communication device (232) may communicate with the server (240) via a long-range communication channel (230). For example, the external communication device (232) may be a mobile phone that communicates with a base station (234) over a long-range communication channel (230), such as a cellular RF channel, and connect to the server (240) over a channel (236). The channel (236) may be a land line, cellular line or other communication channel, such as the Internet. In some embodiments, the external communication device (232) may be a satellite communication device capable of communicating with the server (240) from anywhere around the world. The server (240) may constitute a medical center, hospital and the like, as well as any computers, hospital equipment and human personnel located at any such facility.

In some embodiments, the server (240) may communicate with a rescue team (250) (e.g., a medical team, paramedics and/or an ambulance) over the channel (236) (e.g., land or cellular lines) and direct the rescue team (250) to the location of the patient (210). In some embodiments, the external communication device (232) may communicate directly with the rescue team (250).

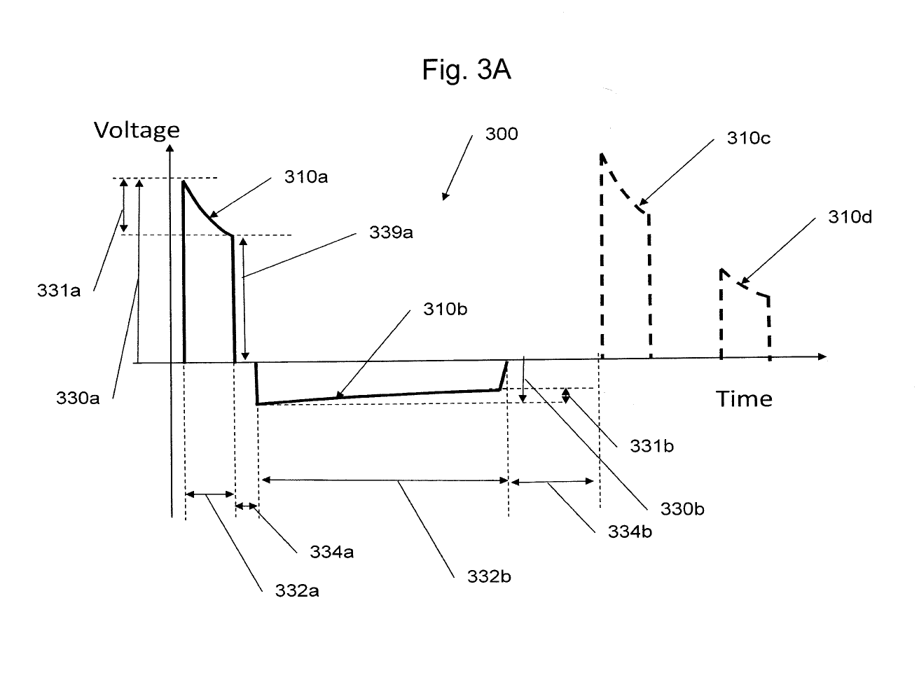

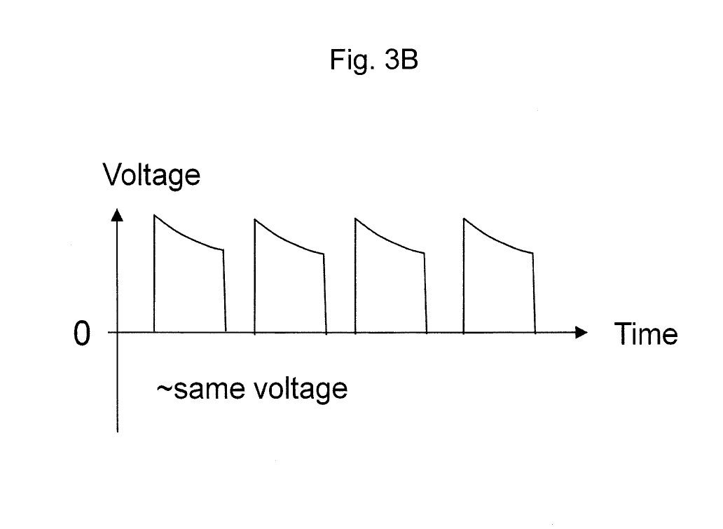

FIGS. 3A and 3B show pulse waveform configurations produced by delivering pulses to the heart according to the subject matter of the present disclosure. The pulses may be of the same or opposite polarity, of similar, shorter or longer duration and/or of the same, higher or lower voltage. Each pulse may be characterized by initial voltage, duration, voltage drop and/or dwell time (unless the pulse is the last in the pulse train). Other pulse parameters, such as pulse rise time, fall time and ringing, may also be considered and may be influenced by the induction of the pulse circuitry, including without limitation, the electrode leads and the types of switches used in the pulse generation circuitry. When more than two electrodes are used for delivering a pulse, waveforms on different electrodes may differ. A first pulse may be delivered between one pair of electrodes while another pulse or pulses may be delivered between a different pair of electrodes. The different pair of electrodes may have one electrode in common. In some embodiments, three or more electrodes may be used for delivering the same pulse. However, voltage and current may be unevenly divided among the electrodes in a multi-electrode (more than two electrodes) pulse.

In some embodiments, a flexible pulse train generator may be used that allows generation of a pulse train waveform according to the present disclosure. A flexible pulse train generator may be used that provides for the selection of a pulse train waveform from among two or more alternatives. In some embodiments, a pulse train waveform may be tailored to a patient and/or his or her medical condition at the time of the pulse delivery. In addition to known individual variations in the pulse energy required for successful cardioversion, there may be individual variations in the perception of pain or discomfort caused by these pulses. In some embodiments, a default waveform may be used first and, if the default pulse train fails to defibrillate, a second train with different time and energy characteristics may be determined to be more efficacious. In some embodiments, waveforms and other pulse parameters may be tested at a medical facility on a patient and a waveform that efficiently defibrillates, yet results in tolerable discomfort, could be selected and used for future defibrillations.

The use of several pulses of high voltage may be more efficient than delivering the same charge or the same energy in a form of one decaying pulse, since in this waveform, the tissue is subjected to higher voltage throughout the application of the voltage. Increased defibrillation efficiency may be advantageous for several reasons. For example, the volume of capacitors used to store electrical energy depends on the maximum possible stored energy. Therefore, reducing the needed energy enables reducing the size of the capacitors and the size of the defibrillator. Similarly, smaller batteries may be used. Additionally, increased defibrillation efficiency may lead to reduced pain or discomfort associated with defibrillation. In addition, the use of two consecutive short pulses with an interval between them could lead to a reduction of pain since the second pulse could be set to stimulate the chest muscle and the nerves at the refractory period of the cells excited by the first pulse, thereby resulting in a reduced chest muscle contraction and nerve response and reduced pain or discomfort.

FIG. 3A shows a pulse train (300) having a first pulse (310a) and a second pulse (310b). The first pulse (310a) may have an initial voltage (330a), a voltage drop (331a), a pulse duration (332a) and a dwell time (334a). The voltage drop (331a) may be determined by the capacitance holding the charge for the first pulse (310a), the pulse duration and the current that is influenced by the impedance of the tissue of the heart. FIG. 3A also shows a pulse train (300a) having a first pulse (310c) and a second pulse (310d) with a predetermined interval (310e) between them. The interval (310e) may be 0-150 milliseconds. In some embodiments, the total length of the pulse train (300a) should not exceed the individual cardiac refractory period, thus eliminating the possibility that the train would induce ventricular fibrillation as triggered by the R-wave.

Generating a train of pulses having (i) a second pulse with an initial voltage (330b) larger than the final voltage (339a) of the first pulse after the second pulse voltage has dropped or (ii) a second pulse with an initial voltage (330b) that is equal to or larger than the final voltage (339a) of the first pulse, requires using a pulse generator capable of compensating for the voltage drop during the first pulse. An exemplary pulse generator is disclosed in International Patent Application No. PCT/US2011/036828, filed on May 17, 2011 and entitled “Configurable Pulse Generator,” the disclosure of which is incorporated herein by reference.

FIG. 3B shows a monophasic pulse train having more than three pulses with the same polarity and similar voltage and duration. In some embodiments, at least two of the four pulses may be delivered by a different configuration of electrodes. A first pulse may be delivered between a first pair of electrodes (e.g., 123a and 123b in FIG. 1), a second pulse may be delivered between a second pair of electrodes (e.g., 125a and 125b in FIG. 1), a third pulse may be delivered between a third pair of electrodes (e.g., 123a and 125b in FIG. 1) and a fourth pulse may be delivered between a fourth pair of electrodes (e.g., 123b and 125a in FIG. 1).

In some embodiments, the pulse trains may be produced such that the net charge delivered to the heart muscle during a defibrillation attempt is zero. That is, the total charge delivered to the heart during one phase is neutralized by the charge taken from the heart by the portion of the pulse train that is in the opposite polarity. Net charge waveforms need to be at least biphasic. In triphasic or other non-symmetric waveforms, the charge delivered in each phase needs to be calculated and adjusted accordingly. In some embodiments, delivered charge is measured by the defibrillator to adjust the waveform such that zero or near-zero net charge would be delivered. For example, the last pulse in the train may be adjusted to compensate for the net charge delivered in the preceding pulses. Zero or near-zero net charge pulse trains may be used with waveforms that are not mono-polar. In some embodiments, a pulse train may start with 20-microsecond pulses and include one of monophasic, biphasic and/or triphasic pulse sequences after the first pulse.

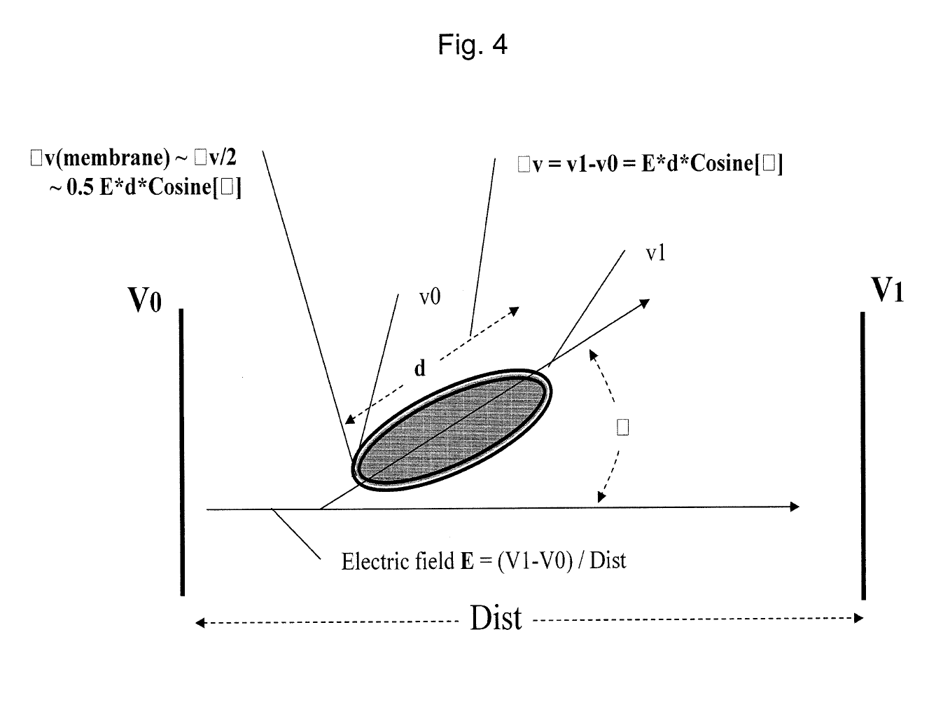

The subject matter of the present disclosure is also directed to embodiments that use more than one electrode pair to defibrillate the heart to increase defibrillation efficiency. More specifically, successful defibrillation involves activating all or at least a majority (e.g., over 50% or 90%) of the heart's muscle cells. While electric current generally flows through the somewhat conductive extracellular liquid, cell membranes are generally non-conductive when the cell is not activated. To activate a cell, the electric field applied across the membrane of each cell should be above a certain threshold. Heart cells are elongated and may be oriented at an angle with respect to the local direction of the electric field caused by an pulse delivered to the heart. The potential difference becomes a function of both the strength of the electric field and the angle between the longitudinal axis of each cell and the local electric field in its vicinity. For example, referring to FIG. 4, when an electric field, E, is oriented at an angle, θ, to the longitudinal axis of a cell having a length, d, the induced potential difference on the cell's membrane may be approximated using the equation, Δv (membrane) ˜0.5*E*d*cosine [θ]. To ensure activation, an electric field, E, large enough to activate even cells with unfavorable angular orientation may be used.

Using a plurality of pulses, each having an electric field, E, oriented at a different angle, surrounds and/or exposes the heart to more defibrillation energy and thus increases the probability of successful defibrillation and allows for lower-energy pulses to be used, thereby reducing the pain and/or discomfort potentially associated with those pulses. Other mechanisms may be acting to determine the angular dependency of the sensitivity of a cardiac cell or a group of cells to an electric field vector. Regardless of the exact mechanism, it may be advantageous to have the vector of the electric field parallel, or close to parallel, to the most sensitive cellular axis of the cardiac tissue. As different cells or parts of the tissue may have different orientations of the most sensitive field directions, lower defibrillation thresholds may be achieved by providing a plurality of pulses having different electric field directions.

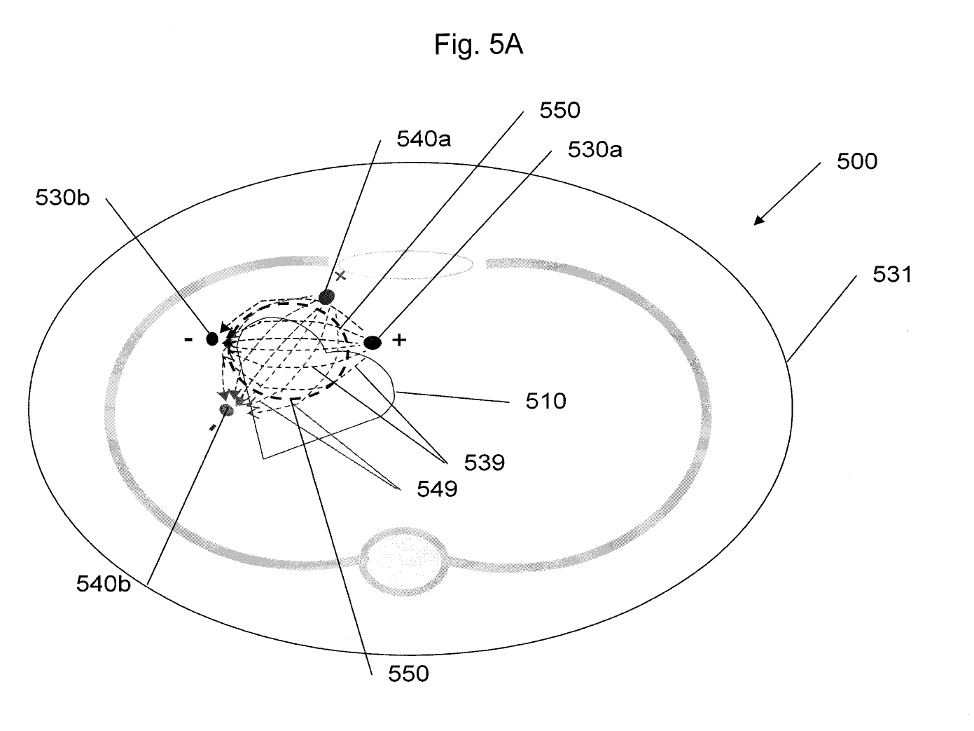

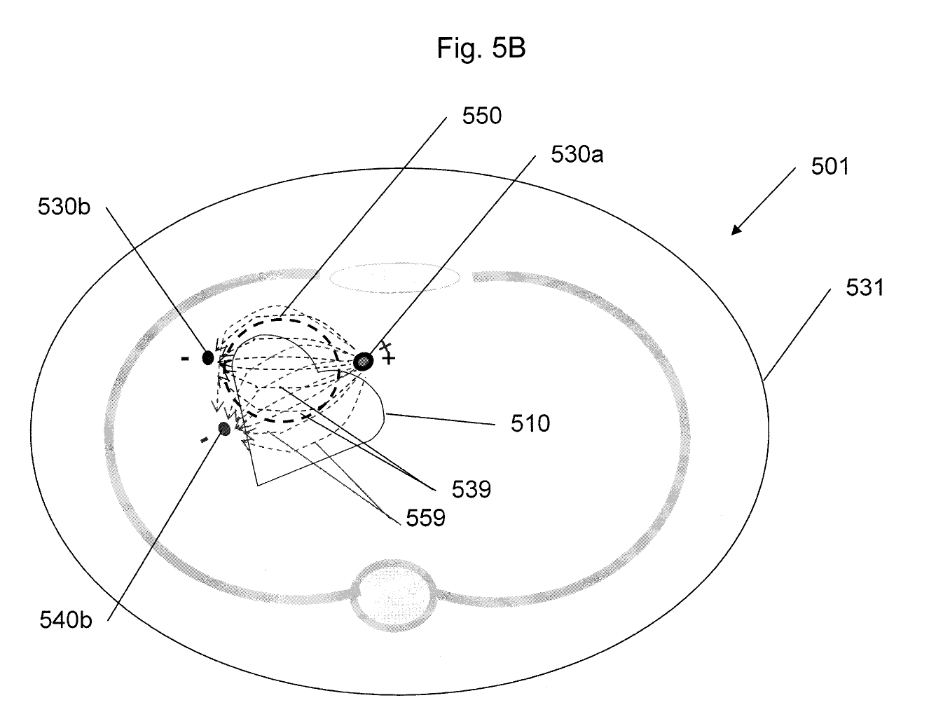

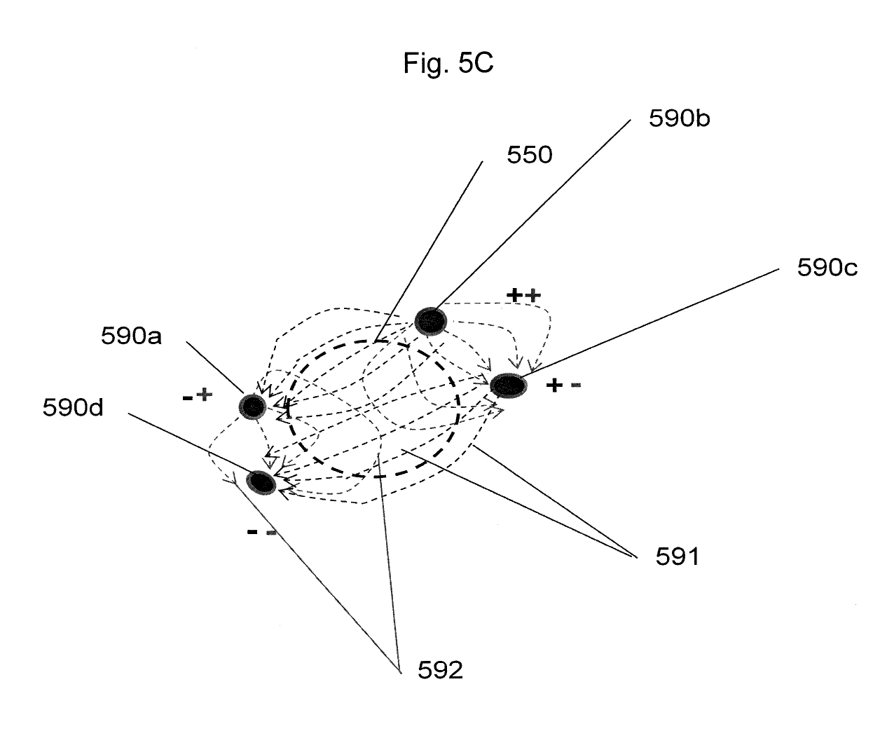

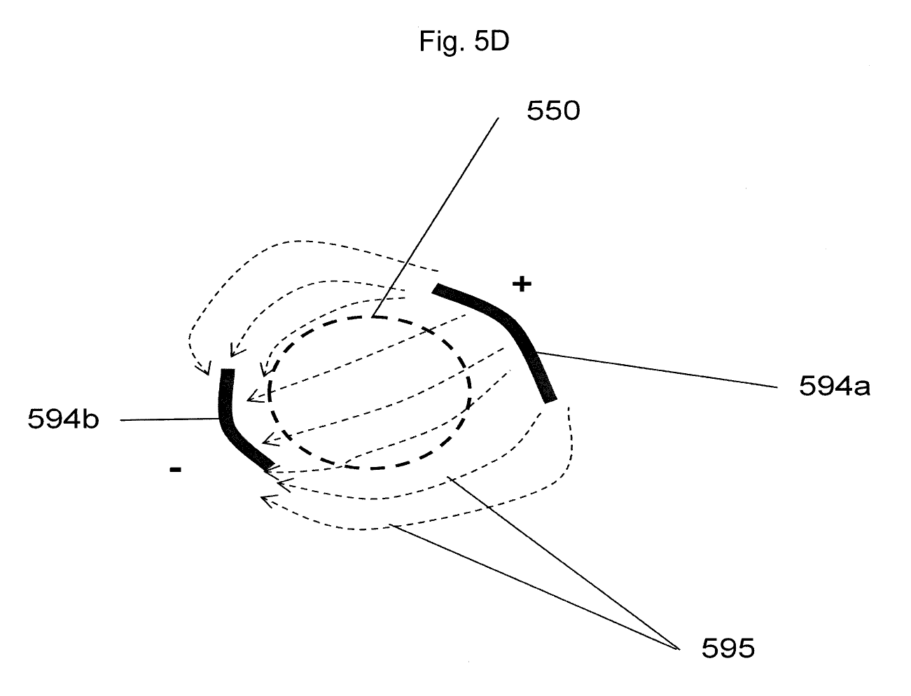

FIGS. 5A-5C show electric fields provided at different angles to each other using three electrodes or more according to some embodiments of the present disclosure. FIG. 5A shows a cross-section of a patient's torso (531). Four defibrillation electrodes (530a), (530b), (540a) and (540b) are positioned in, on and/or around a heart (510). FIG. 5A shows electrodes (530a) and (530b) forming a first pair of electrodes and electrodes (540a) and (540b) forming a second pair of electrodes. A defibrillator (not shown) may apply a voltage to the first pair of electrodes (530a) and (530b) and the second pair of electrodes (540a) and (540b) sequentially. A first voltage, V₁, may be applied to the first pair of electrodes (530a) and (530b) to form a first electric field (539). The electrode (530a) may be positively charged and the electrode (530b) may be negatively charged or vice versa. After a predetermined period of time, which may be shorter, equal to or longer than the duration of the first voltage, V₁, the first voltage, V₁, may be removed and a second voltage, V₂, may be applied to the second pair of electrodes (540a) and (540b) to form a second electric field (549). The electrode (540a) may be positively charged and the electrode (540b) may be negatively charged or vice versa. As shown in FIG. 5A, the first electric field (539) and the second electric field (549) are not parallel at any point. By positioning the electrodes (530a), (530b), (540a) and (540b) in certain locations in and around the heart (510), the first electric field (539) and second electric field (549) may be at a large angle to each other to sufficiently cover the heart (510) with defibrillating pulses so as to activate all or at least a majority (e.g., over 50% or 90%) of the heart's muscle cells.

FIG. 5B also shows a cross-section of a patient's torso (531) with three electrodes (530a), (530b) and (540b) positioned in, on and/or around the heart (510), wherein electrode (530a) acts as a common electrode. Electrode (530a) and electrode (530b) may form a first pair of electrodes and electrodes (530a) and (540b) may form a second pair of electrodes. A defibrillator (not shown) may apply a first voltage, V₁, to the first pair of electrodes (530a) and (530b) to form a first electric field (539). The electrode (530a) may be positively charged and the electrode (530b) may be negatively charged or vice versa. After a predetermined period of time, which may be shorter, equal to or longer than the pulse duration of the first voltage, V₁, the first voltage, V₁, may be removed and a second voltage, V₂, may be applied to the second pair of electrodes (530a) and (540b) to form a second electric field (559). The electrode (530a) may be positively charged and the electrode (540b) may be negatively charged or vice versa. In some embodiments, the common electrode (530a) may serve as a positively-charged electrode during the application of the first voltage, V₁, and a negatively-charged electrode during the application of the second voltage, V₂. As shown in FIG. 5B, the first electric field (539) and the second electric field (559) are not parallel at any point. By positioning the electrodes (530a), (530b) and (540b) in certain locations in, on and around the heart (510), the first electric field (539) and second electric field (559) may be at a large angle to each other to sufficiently cover the heart (510) with defibrillating pulses so as to activate all or at least a majority (e.g., over 50% or 90%) of the heart's muscle cells.

FIG. 5C shows electric fields provided at different angles among four electrodes (590a, 590b, 590c, 590d). In some embodiments, a positive voltage may be applied to electrode (590b) and electrode (590c) and a negative voltage may be applied to electrode (590a) and electrode (590d). These voltages are shown in FIG. 5C as the leftmost “+” or “−” in the pair of signs adjacent to each of the reference numerals (590a), (590b), (590c) and (590d). As a result, a first electric field (591) is provided between (590a) and (590b) and between (590c) and (590d), as shown in FIG. 5C. After a predetermined period of time, which may be shorter, equal to or longer than the duration of the first positive and negative voltages, a positive voltage may be applied to electrode (590a) and electrode (590b) and a negative voltage may be applied to electrode (590c) and electrode (590d). These voltages are shown in FIG. 5C as the rightmost “+” or “−” in the pair of signs adjacent to each of the reference numerals (590a), (590b), (590c) and (590d). As a result, a second electric field (592) is provided between (590a) and (590d) and between (590b) and (590c). As shown in FIG. 5C, the first electric field (591) and the second electric field (592) are not parallel at any point.

By positioning the electrodes (590a), (590b), (590c) and (590d) at certain locations in, on and/or around heart relative to each other, the angle between the first electric field (591) and the second electric field (592) may be variably adjusted within a defibrillation zone (550) of the cardiac tissue. In some embodiments, it may be advantageous to maintain an electric field great enough to affect defibrillation in a defibrillation zone (550) while minimizing the electric field in pain-sensitive areas outside the defibrillation zone (550). In some embodiments, the positive and negative voltage potential applied to the electrodes (590a), (590b), (590c) and (590d) during a pulse may or may not be equal. By selecting different voltage potential on the electrodes in various embodiments, the strength and orientation of the electric field may be manipulated. Depending on the configuration of the electrodes in, on and/or around the art, the electric field in the defibrillation zone (550) may be more uniform in intensity and/or orientation. In some embodiments, the average and/or minimal angle between the first electric field (591) and the second electric field (592) may be larger. According to some embodiments of the present disclosure, using an electrode configuration similar to that shown in FIG. 5C may also provide for electric fields that are more confined to a certain defibrillation zone.

Electric fields may be provided between two or more extended electrodes according to some embodiments of the present disclosure. FIG. 5D shows an exemplary embodiment having a first extended electrode (594a) and a second extended electrode (594b). One or both of the extended electrodes (594a, 594b) may have an elongated shape and may be positioned along one or more of the leads and/or sub-leads of the IAD (100) of the present disclosure. In some embodiments, more than two extended electrodes may be employed. In some embodiments, the extended electrodes may be employed alone or together with one or more point electrodes. The extended electrodes (594a) and (594b) may have the same or different length, width and/or height dimensions and may be positioned at different latitudes and/or longitudes in or around the heart. In some embodiments, a positive voltage may be applied to the first electrode (594a) and a negative voltage may be applied to the second electrode (594b) to form an electric field (595) therebetween. As seen in FIG. 5D, the resulting lines of electric field (595) compared to those of the point electrode configurations, for example, as shown in FIGS. 5A-5C, are more uniform within the defibrillation zone (550). It should be noted, however, that FIG. 5D shows the electric field (595) in two dimensions and that a three-dimensional view of electric field (595) is more complex. Furthermore, in some embodiments, voltage potential along an extended electrode may vary. For example, a segmented extended electrode may be used and the voltage along each segment may be separately controlled. In some embodiments, the extended electrodes may include resistive elements configured to control current and/or voltage along the electrode.

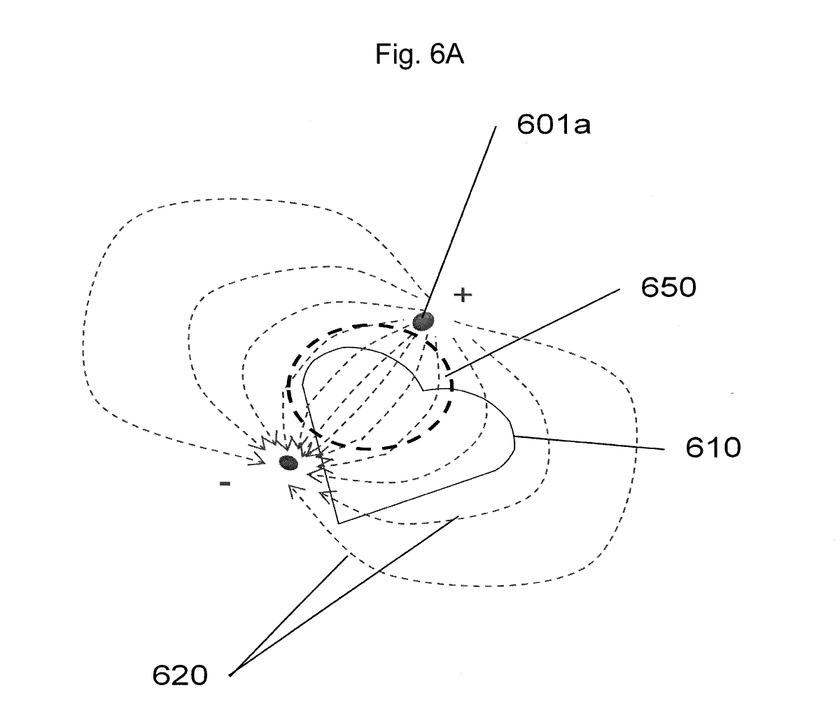

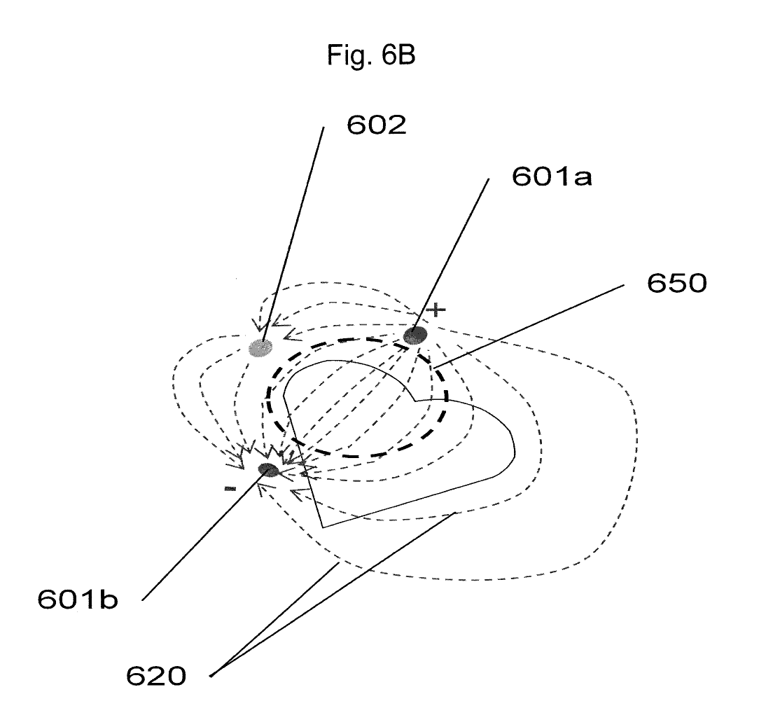

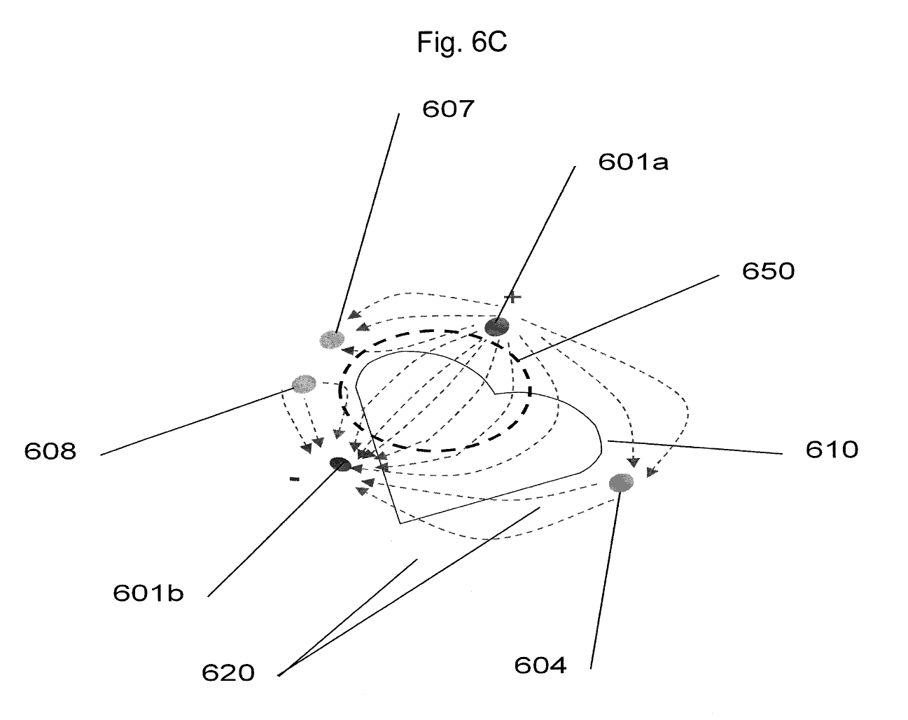

Some embodiments of the present disclosure may be directed to selectively confining or otherwise controlling the distribution of one or more electric fields provided in, on and/or around the heart. FIG. 6A shows an electric field (620) distributed between a positively-charged electrode (601a) and a negatively-charged electrode (601b) for defibrillating one or more chambers of the heart (610). In FIG. 6A, the electrode (601b) is depicted as an electric field containment electrode in that it attracts positive charge from electrode (601a) to form and confine the electric field (620) to the area of the heart (610) and, in particular, a defibrillation zone (650). FIG. 6B is similar to FIG. 6A but includes an electric field confinement electrode (602) positioned toward the upper left side of the heart (610) that bends, shapes or otherwise alters the lines of the electric field (620) by further confining a portion of the electric field (620) to the area immediately surrounding the heart (610) and creating a greater density of the electric field (620) in and around the defibrillation zone (550). In some embodiments, the voltage potential of the electric field confinement electrode (602) may be selected to be between the voltage potentials of the electrode (601a) and the electrode (601b). The existence of the electric field confinement electrode (602) bends the field lines in the vicinity of the electric field confinement electrode (602) and causes more confinement of the field within the defibrillation zone (650).

In some embodiments of the present disclosure, more than one confinement electrode may be used. FIG. 6C shows a first electric field confinement electrode (604), a second electric field confinement electrode (607) and a third electric field confinement electrode (608) employed in conjunction with the electrode (601a) and the electrode (601b). In some embodiments, the first electric field confinement electrode (604) may be configured to bend, shape or otherwise alter the electric field (620) in the lower right side of the heart (610) in a manner similar to the electric field confinement electrode (602) positioned toward the upper left side of the heart (610) shown in FIG. 6B. The second electric field confinement electrode (607) and third electric field confinement electrode (608) may be positioned toward the upper left side of the heart (610) and used to bend, shape or otherwise alter the electric field (620) in that area. In some embodiments, the same or similar voltage may be applied to the second electric field confinement electrode (607) and third electric field confinement electrode (608) and be equal to or near the mid-point between the voltages of the electrode (601a) and the electrode (601b), in which case there will be little or no electric field between the second electric field confinement electrode (607) and third electric field confinement electrode (608). While FIG. 6C shows the electric field (620) in two dimensions, the three-dimensional view of the field (620) is more complex.

Some embodiments of the present disclosure seek to provide electrode configurations and voltage distributions that remove or otherwise minimize the electric field of a pulse in tissue areas that are prone to discomfort during defibrillation, while at the same time providing electric fields having strengths in relevant cardiac tissue areas sufficient to successfully defibrillate. In traditional electrode arrangements of implanted atrial defibrillator systems, as exemplified in FIG. 1 of U.S. Pat. No. 5,282,837 to Adams et al., filed on Apr. 12, 1991 and entitled “Atrial Defibrillator and Method,” electrodes are positioned at large distances from each other and require high voltages to achieve effective defibrillation. As a result, the electric field lines resulting from these high voltages between two distanced electrodes extend far beyond the relevant cardiac tissue areas that needed to be defibrillated, e.g., the left and/or right atrium. Because the pain and discomfort associated with defibrillation is partially due to nerve stimulation and/or muscle contraction in response to electrical stimulation, electric fields that extend beyond the relevant cardiac tissue contribute to discomfort without contributing to defibrillation.

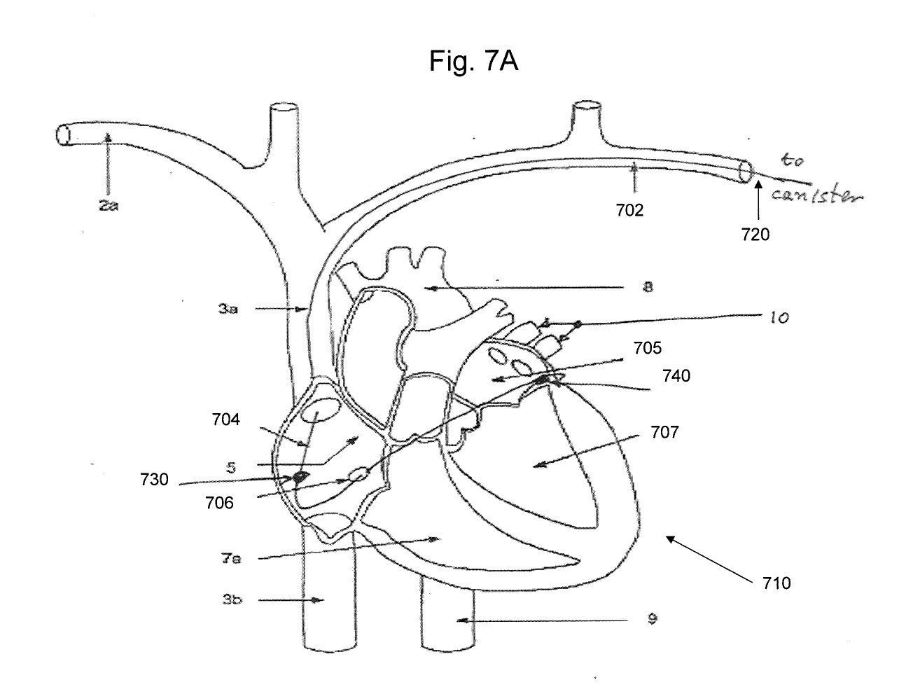

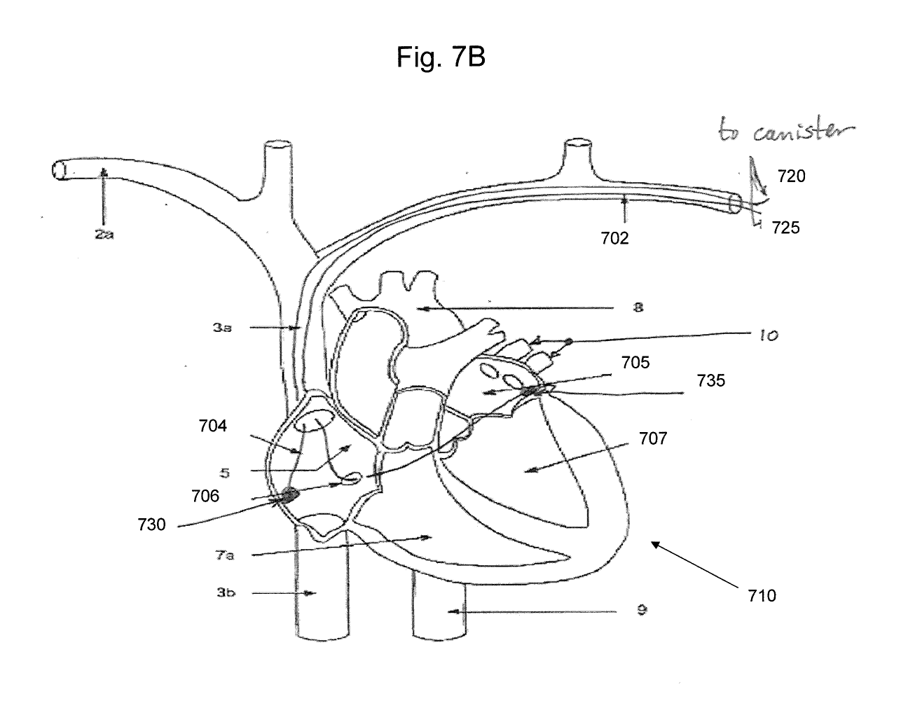

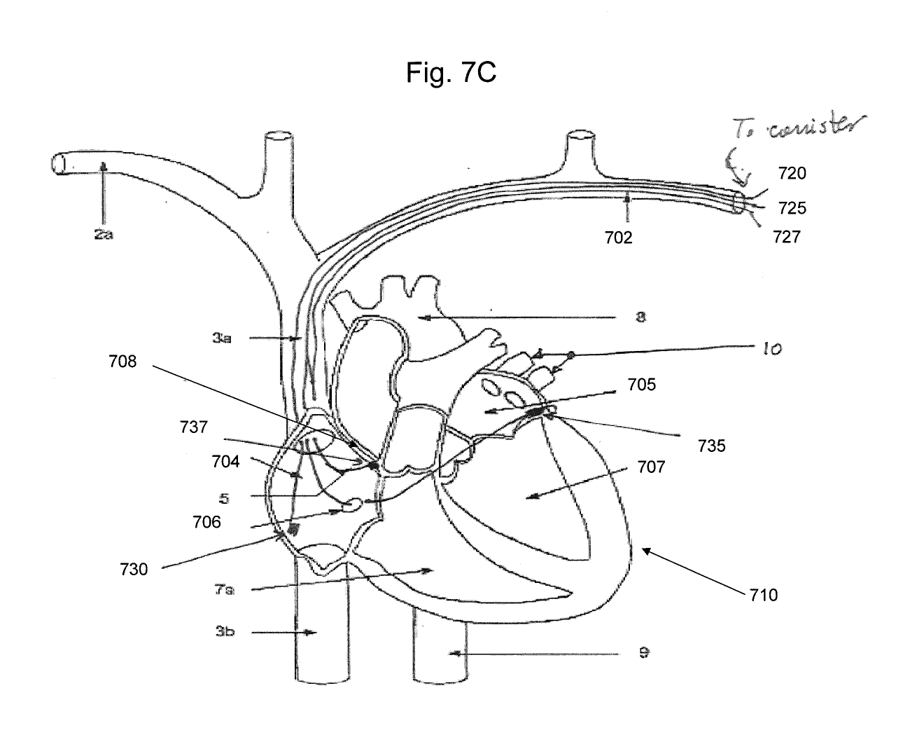

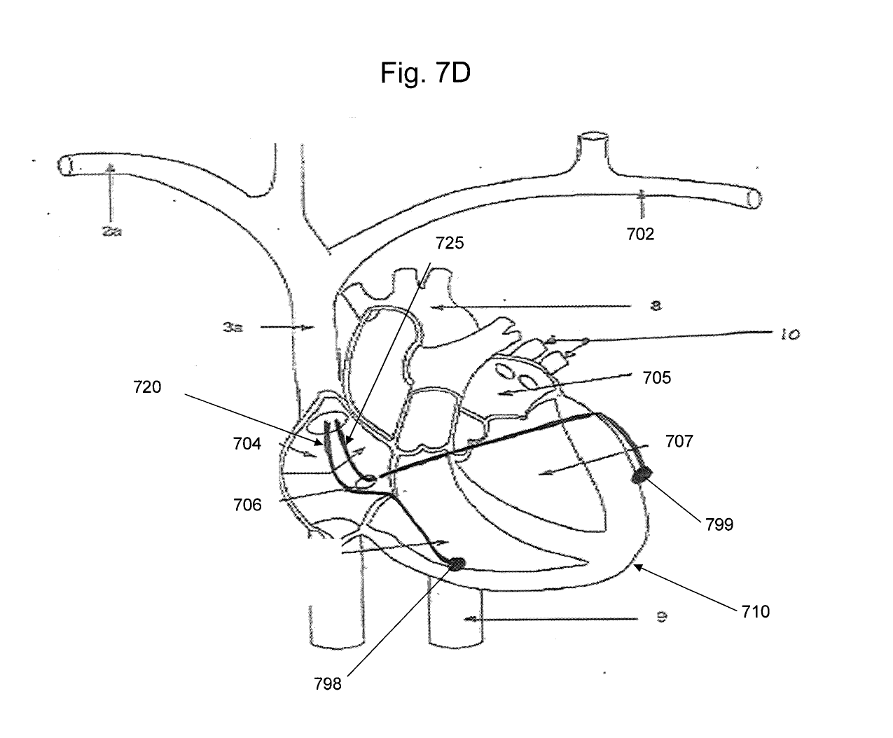

FIGS. 7A-7C show electrode configurations according to the subject matter of the present disclosure that minimize electric field discomfort during effective defibrillation. FIG. 7A shows a single lead (720) entering a heart (710) through the left subclavian vein (702), entering the right atrium (704) and reaching a distal location on the left side of the heart (710) and proximal to the septum between the left atrium (705) and the left ventricle (707) via the coronary sinus (706). The single lead (720) may have a first electrode (730) positioned in the right atrium (704) of a heart (710) and a second electrode (740) positioned inside the coronary vein (not shown) of the heart (710).

FIG. 7B shows two single leads, a first single lead (720) and a second single lead (725), entering the heart (710) through the left subclavian vein (702) and entering the right atrium (704). The first single lead (720) terminates in the right atrium (704) and has a first electrode (730) positioned in the right atrium (704). The second single lead (725) traverses to a location on the left side of the heart (710) and proximal to the septum between the left atrium (705) and the left ventricle (707) via the coronary sinus (706). The second single lead (725) has a first electrode (735) positioned inside the coronary vein (not shown).

FIG. 7C shows three single leads, a first single lead (720), a second single lead (725) and a third single lead (727), entering the heart (710) through the left subclavian vein (702) and entering the right atrium (704). The first single lead (720) terminates in the right atrium (704) and has a first electrode (730) positioned in the right atrium (704). The second single lead (725) traverses to a location on the left side of the heart (710) and proximal to the septum between the left atrium (705) and the left ventricle (707) via the coronary sinus (706). The second single lead (725) has a first electrode (735) positioned inside the coronary vein (not shown). The third single lead (727) terminates in the right atrium (704) and has a first electrode (737) positioned in the inter-atrial septum (708).

FIG. 7D shows two single leads, a first single lead (720) and a second single lead (725), entering the heart (710) through the left subclavian vein (702) and entering the right atrium (704). The first single lead (720) terminates in the right ventricle and has a first electrode (798) positioned in the right ventricle for ventricular defibrillation. The second single lead (725) traverses to the left side of the heart (710) via the coronary sinus (706) and has a first electrode (799) positioned in the descending azygous vein for ventricular defibrillation.

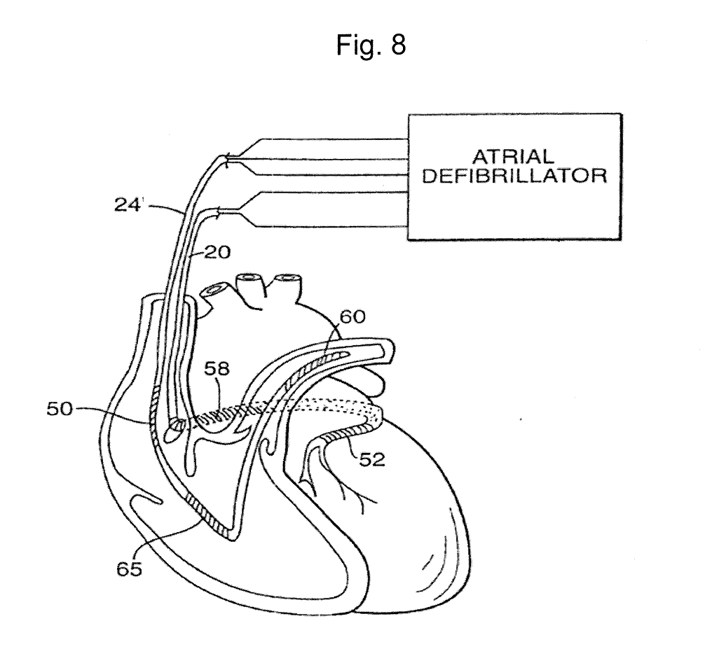

FIG. 8 shows a depiction of FIG. 1 from U.S. Pat. No. 6,327,500 to Cooper et al., filed on Nov. 15, 1999 and entitled “Dual Shock Atrial Defibrillation Apparatus,” and is incorporated herein by reference in its entirety. This patent discloses electrodes (50), (52), (58), (60) and (65) positioned on leads (20) and (24) in various locations within the heart. The lead configurations disclosed in the patent may be adopted herein to position elongated, segmented or small length electrodes.

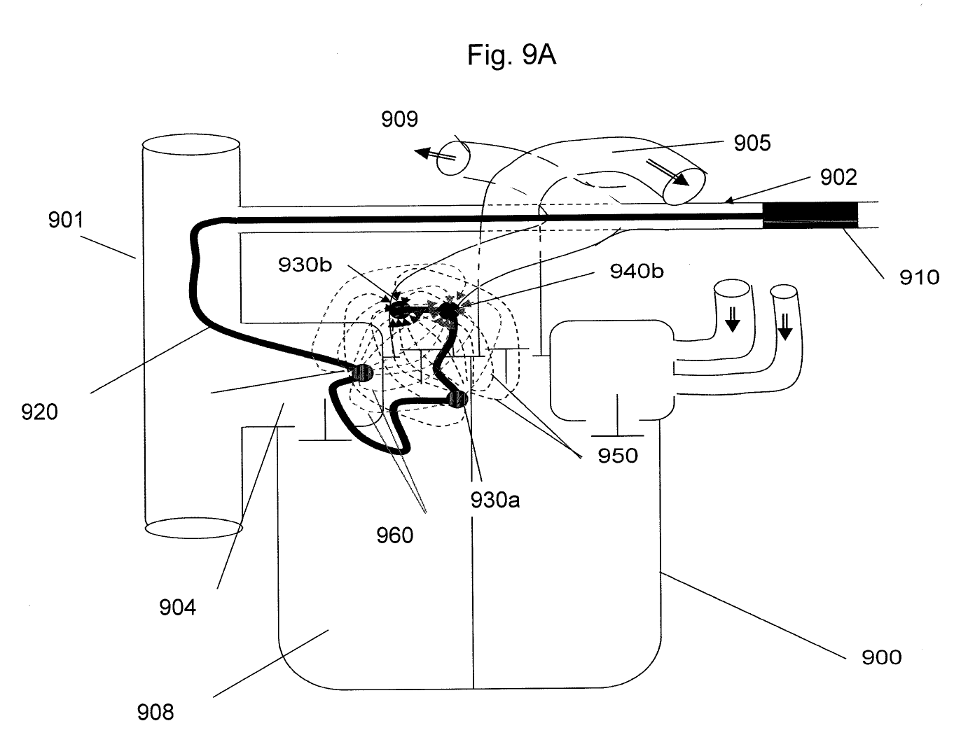

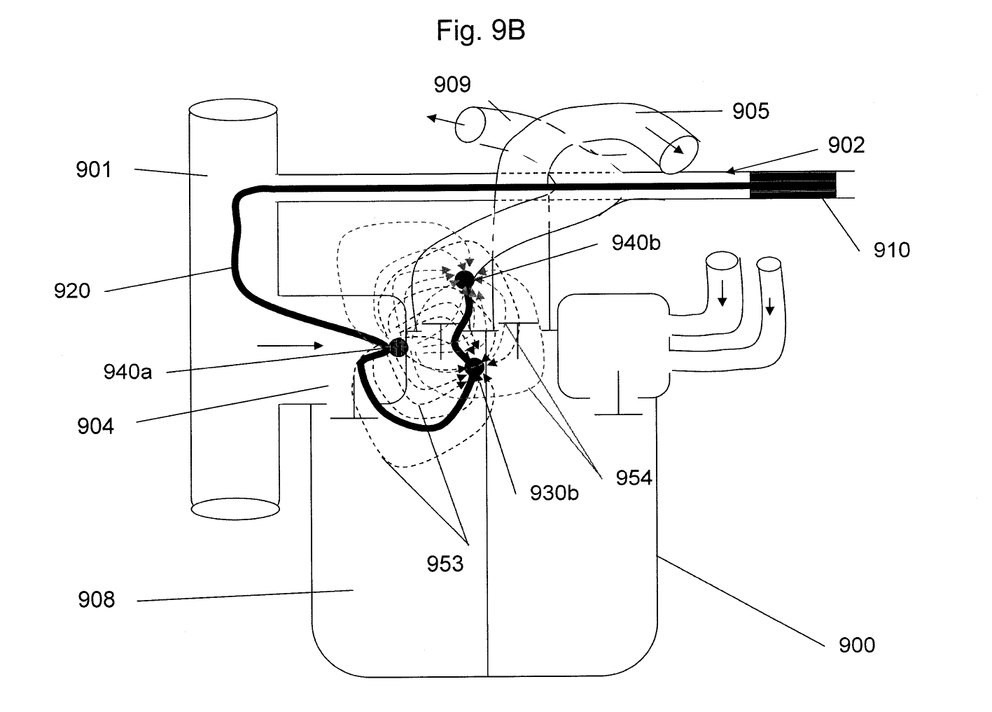

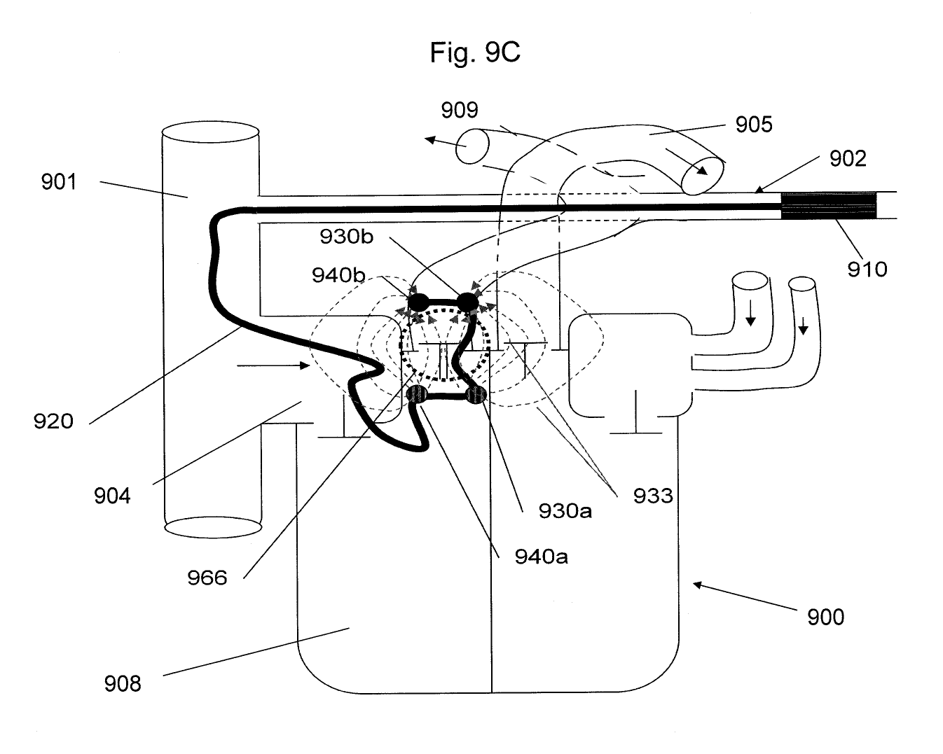

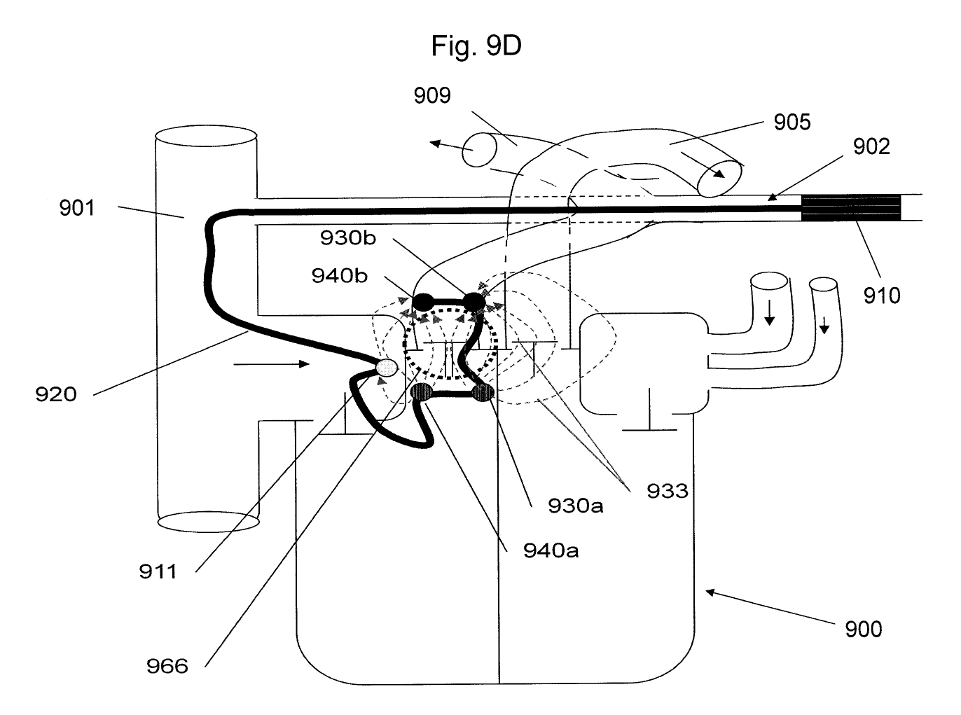

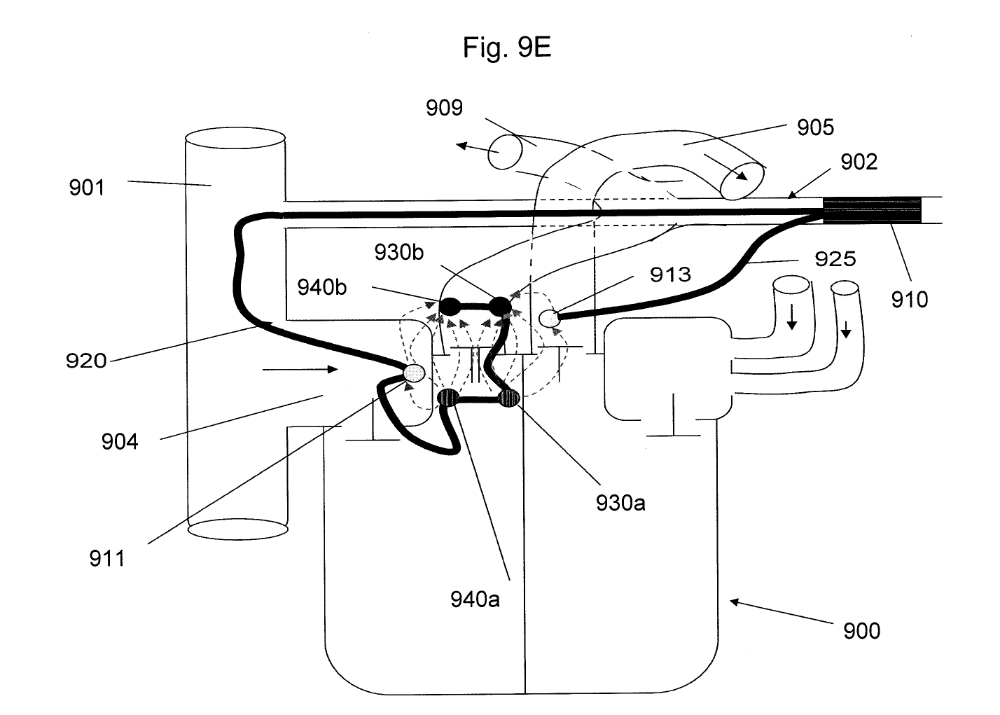

Embodiments of the present disclosure may also be directed to methods for reducing the pulse voltage and the energy required to successfully defibrillate the left and/or right atrium. In some embodiments, methods may involve reducing discomfort associated with atrial defibrillation and, in particular, by concentrating the electric field to a specific localized area of the heart. Embodiments of the present disclosure that are directed to these methods are provided in FIGS. 9A-9E and, in particular, implement configurations described above with respect to FIGS. 5A-5D and 6A-6C. In some of these embodiments, the focal point of the atrial fibrillation may be confined within a specific area of the heart and identified during a medical examination and/or by collecting ECG data, for example, from a plurality of electrodes using sensing electronics (112) and/or data analysis by controller (113), as shown in FIG. 1. In some embodiments, a defibrillation pulse or a train of defibrillation pulses may target the focal point of the atrial fibrillation by, for example, appropriately positioning one or more electrodes in, on and/or around the heart relative to the focal point location.

FIG. 9A shows a configuration of four electrodes placed in, on and/or around the heart (900) according to some embodiments of the present disclosure. The four electrodes are positioned along a single lead (920) that is connected to a main body (910) of an IAD located inside the subclavian vein (902) of the heart (900). The single lead (920) enters the right atrium (904) through the vena cava (901) and then enters the right ventricle (908) and into the pulmonary artery (909). The main body (910) may be implanted elsewhere in the body of a patient, for example, in the upper anterior portion of the chest, anterior to the pectoralis major muscle or any other suitable location. In some embodiments, the main body (910) may be used as a defibrillation electrode and/or as a grounding electrode for one or more ECG sensing electrodes. As shown in FIG. 9A, the four electrodes may be positioned in and around the right atrium (904) and at or near the connection of the right ventricle (908) with the pulmonary artery (909). In some embodiments, an electrode (930a) may be positioned in the right ventricle (908), an electrode (930b) and an electrode (940b) may be positioned in pulmonary artery (909) and an electrode (940a) may be positioned in the right atrium (904). In some embodiments, electrode (930a) and electrode (930b) may for a first defibrillation pair and electrode (940a) and electrode (940b) may form a second defibrillation pair.

As shown in FIG. 9A, an electric field (950) is provided between the first defibrillation pair of electrode (930a) and electrode (930b) when a first pulse is applied and an electric field (960) is provided between the second defibrillation pair of electrode (940a) and electrode (940b) at an angle to the electric field (950) when a second pulse is applied. This configuration of the electric field (950) and electric field (960) intersecting at an angle to each other provides a concentrated electric field in the right atrium (904). In some embodiments, the order of pulses may be reversed, such that a first pulse may be applied to electrode (940a) and electrode (940b) and a second pulse may be applied to electrode (930a) and electrode (930b). In some embodiments, the location of the electrodes may be different, the electrodes may be paired differently and/or the polarity of the pulses may be different. In some embodiments, more than two pulses may be used, for example, a first pulse may be applied between electrode (930a) and electrode (930b), a second pulse may be applied between electrode (940a) and electrode (940b) and a third pulse may be applied between electrode (930a) and electrode (940a). Using different and numerous permutations of electrode pairings may provide more orientations to the electric field vectors and lead to more effective but less painful defibrillation. In some embodiments, one or more electrodes may be added in the azygous vein to support ventricular defibrillation.

FIG. 9B shows a configuration of three electrodes placed in, on and/or around the heart (910) according to some embodiments of the present disclosure. The three electrodes are positioned along a single lead (920) that is connected to a main body (910) of an IAD located inside the subclavian vein (902) of the heart (900). The single lead (920) enters the right atrium (904) through the vena cava (901) and then enters the right ventricle (908) and into the pulmonary artery (909). The main body (910) may be implanted elsewhere in the body of a patient, for example, in the upper anterior portion of the chest, anterior to the pectoralis major muscle or any other suitable location. In some embodiments, the main body (910) may be used as a defibrillation electrode and/or as a grounding electrode for one or more ECG sensing electrodes. As shown in FIG. 9B, the three electrodes may be positioned in and around the right atrium (904) and at or near the connection of the right ventricle (908) with the pulmonary artery (909). In some embodiments, an electrode (930b) may be positioned in the right ventricle (908), an electrode (940b) may be positioned in pulmonary artery (909) and an electrode (940a) may be positioned in the right atrium (904). In some embodiments, electrode (940a) may be used as a common electrode whereby electrode (930b) and electrode (940a) may for a first defibrillation pair and electrode (940a) and electrode (940b) may form a second defibrillation pair.

As shown in FIG. 9B, an electric field (953) is provided between the first defibrillation pair of electrode (930b) and electrode (940a) when a first pulse is applied and an electric field (954) is provided between the second defibrillation pair of electrode (940a) and electrode (940b) at an angle to the electric field (953) when a second pulse is applied. This configuration of the electric field (953) and electric field (954) intersecting at an angle to each other provides a concentrated electric field in the right atrium (904). In some embodiments, the order of pulses may be reversed, such that the first pulse may be applied to electrode (930b) and electrode (940b) and the second pulse may be applied to electrode (930b) and electrode (940a). In some embodiments, the location of the electrodes may be different, the electrodes may be paired differently and/or the polarity of the pulses may be different. In some embodiments, more than two pulses may be used, for example, a first pulse may be applied between electrode (940a) and electrode (930b), a second pulse may be applied between electrode (940a) and electrode (940b) and a third pulse may be applied between electrode (930b) and electrode (940b). Using different and numerous permutations of electrode pairings may provide more orientations to the electric field vectors and lead to more effective but less painful defibrillation. In some embodiments, one or more electrodes may be added in the azygous vein to support ventricular defibrillation.

FIG. 9C shows a configuration for minimizing the magnitude of an electric field outside a defibrillation zone (966) that includes four electrodes placed in, on and/or around the heart (910) according to some embodiments of the present disclosure. The four electrodes are positioned along a single lead (920) that is connected to a main body (910) of an IAD located inside the subclavian vein (902) of the heart (900). The single lead (920) enters the right atrium (904) through the vena cava (901) and then enters the right ventricle (908) and into the pulmonary artery (909). The main body (910) may be implanted elsewhere in the body of a patient, for example, in the upper anterior portion of the chest, anterior to the pectoralis major muscle or any other suitable location. In some embodiments, the main body (910) may be used as a defibrillation electrode and/or as a grounding electrode for one or more ECG sensing electrodes. As shown in FIG. 9C, the four electrodes may be positioned in and around the right atrium (904) and at or near the connection of the right ventricle (908) with the pulmonary artery (909). In some embodiments, an electrode (930a) and an electrode (940a) may be positioned in the right ventricle (908) and an electrode (930b) and an electrode (940b) may be positioned in the pulmonary artery (909). In contrast to embodiments described above with respect to FIGS. 9A and 9B where defibrillation pulses were delivered by one pair of electrodes at a time, embodiments relating to FIG. 9C may include two electrodes being positively charged and two electrodes being negatively charged during the same pulse. For example, in some embodiments, electrode (930a) and electrode (940a) may be similarly or identically charged and electrode (930b) and electrode (940b) may be similarly or identically charged during the delivery of one or more defibrillation pulses.

As shown in FIG. 9C, an electric field (933) is provided between electrode (930a) and electrode (930b) and between electrode (940a) and electrode (940b) when a single defibrillation pulse is applied to the four electrodes at the same time. This configuration provides a concentrated electric field in a defibrillation zone (966) between the electrodes. In some embodiments, the pairing of the electrodes may be different. For example, electrode (940a) and electrode (940b) may be positively charged and electrode (930a) and electrode (930b) may be negatively charged. In some embodiments, the location of the electrodes may be different, the electrodes may be paired differently and/or the polarity of the pulses may be different. In some embodiments, the electrodes may be differently paired in each of two pulses in a pulse train. For example, a first pulse may be delivered when electrode (930a) and electrode (940a) are positively charged and electrode (930b) and electrode (940b) are negatively charge and a second pulse may be delivered when electrode (940a) and electrode (940b) are positively charged and electrode (930a) and electrode (930b) are negatively charged. Using different and numerous permutations of electrode pairings may provide more orientations to the electric field vectors and lead to more effective but less painful defibrillation. In some embodiments, one or more electrodes may be added in the azygous vein to support ventricular defibrillation.

FIG. 9D shows a configuration for minimizing the magnitude of an electric field outside a defibrillation zone (966) that includes five electrodes placed in, on and/or around the heart (910) according to some embodiments of the present disclosure. The five electrodes are positioned along a single lead (920) that is connected to a main body (910) of an IAD located inside the subclavian vein (902) of the heart (900). The single lead (920) enters the right atrium (904) through the vena cava (901) and then enters the right ventricle (908) and into the pulmonary artery (909). The main body (910) may be implanted elsewhere in the body of a patient, for example, in the upper anterior portion of the chest, anterior to the pectoralis major muscle or any other suitable location. In some embodiments, the main body (910) may be used as a defibrillation electrode and/or as a grounding electrode for one or more ECG sensing electrodes. As shown in FIG. 9D, the five electrodes may be positioned in and around the right atrium (904) and at or near the connection of the right ventricle (908) with the pulmonary artery (909). In some embodiments, an electrode (930a) and an electrode (940a) may be positioned in the right ventricle (908), an electrode (930b) and an electrode (940b) may be positioned in the pulmonary artery (909) and a confinement electrode (911) may be positioned in the right atrium. During defibrillation, the voltage on the confinement electrode (911) may be set to a value between the voltage on electrode (930a) and electrode (940a) (which have same or similar voltage) and the voltage on electrodes (930b) and electrode (940b) (which have same or similar voltage). In some embodiments, confinement electrode (911) may bend, shape or otherwise alter electric field (933), as described above with respect to FIG. 9C, to further concentrate the electric field (933) in the defibrillation zone (966) and in turn reduce the electric field in locations away from the defibrillation zone (966) that do not need to be defibrillated. In some embodiments, confinement electrode (911) may be positioned in locations other than the right atrium (904). In some embodiments, one or more electrodes may be added in the azygous vein to support ventricular defibrillation.

FIG. 9E shows a configuration that includes six electrodes placed in or around the heart (910) according to some embodiments of the present disclosure. Five electrodes are positioned along a single lead (920) that is connected to a main body (910) of an IAD located inside the subclavian vein (902) of the heart (900). A sixth electrode is positioned along a second single lead (925) that is also connected to a main body (910) of an IAD located inside the subclavian vein (902) of the heart (900). The single lead (920) enters the right atrium (904) through the vena cava (901) and then enters the right ventricle (908) and into the pulmonary artery (909). The main body (910) may be implanted elsewhere in the body of a patient, for example, in the upper anterior portion of the chest, anterior to the pectoralis major muscle or any other suitable location. In some embodiments, the main body (910) may be used as a defibrillation electrode and/or as a grounding electrode for one or more ECG sensing electrodes. As shown in FIG. 9E, five electrodes may be positioned in and around the right atrium (904) and at or near the connection of the right ventricle (908) with the pulmonary artery (909), while a sixth electrode may be positioned in the aorta (905). In some embodiments, an electrode (930a) and an electrode (940a) may be positioned in the right ventricle (908), an electrode (930b) and an electrode (940b) may be positioned in the pulmonary artery (909), a first confinement electrode (911) may be positioned in the right atrium and a second confinement electrode (913) may be positioned in the aorta (905).

During defibrillation, the voltage on the first confinement electrode (911) and/or the second confinement electrode (913) may be set to a value between the voltage on electrode (930a) and electrode (940a) (which have same or similar voltage) and the voltage on electrodes (930b) and electrode (940b) (which have same or similar voltage). The voltage of the first confinement electrode (911) may be the same as or different from the voltage of the second confinement electrode (913). In some embodiments, the first confinement electrode (911) and the second confinement electrode (913) may bend, shape or otherwise alter electric field (933), as described above with respect to FIG. 9C, to further concentrate the electric field (933) in the defibrillation zone (966) and in turn reduce the electric field in locations away from the defibrillation zone (966) that do not need to be defibrillated. In some embodiments, the first confinement electrode (911) and/or the second confinement electrode (913) may be positioned in locations other than the right atrium (904) and the aorta (905), respectively. The number of field containment electrodes, such as first confinement electrode (911) and second confinement electrode (913), may vary depending on the embodiment and indeed is not limited to two electrodes as shown in FIG. 9E. Similarly, the number of defibrillation electrodes (930a, 930b, 940a, 940b) may be greater than four. In some embodiments, the defibrillation electrodes and/or confinement electrodes may be extended electrodes spanning a length of the lead, as shown in FIG. 5D. In some embodiments, one or more electrodes may be added in the azygous vein to support ventricular defibrillation.

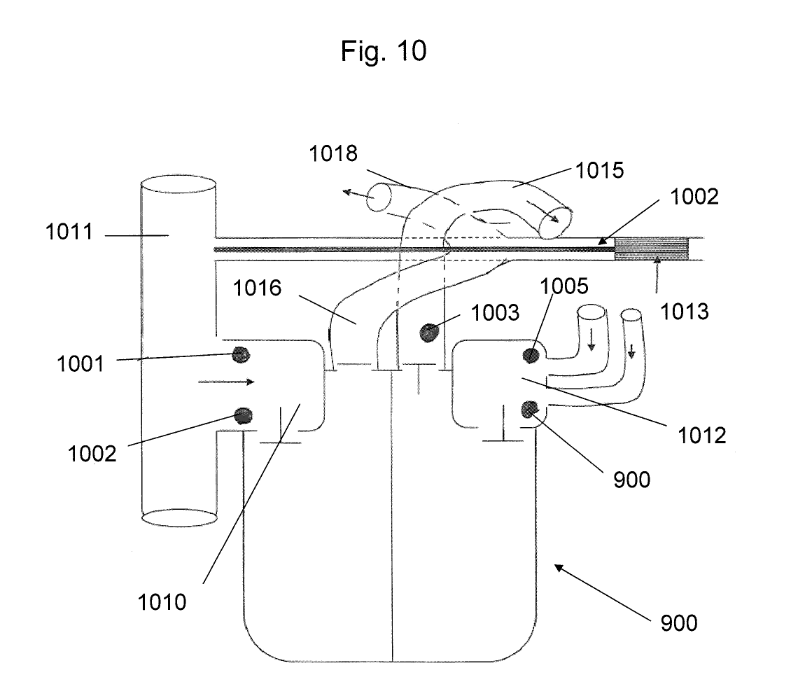

FIG. 10 shows a configuration of electrodes for atrial defibrillation according to the present disclosure. A first right electrode (1001) and a second right electrode (1002) may be positioned in the right atrium (1010) and first left electrode (1004) and a second left electrode (1005) may be positioned in a vein located in the wall of the left atrium (1012), for example, in the lateral heart underneath the left atrial appendage. In some embodiments, a central electrode (1003) may be positioned in the atrial septum (1014), the aorta (1015) or in the pulmonary artery (1018) adjacent to the pulmonary valve (1016). Each electrode may have a separate lead or a lead may carry two, three or more electrodes. In some embodiments, as discussed above with respect to FIGS. 6B, 6C, 9D and 9E, one or more additional electrodes may be used as confinement electrodes to contain the electric field. In some embodiments, one or more electrodes may be added in the azygous vein to support ventricular defibrillation.

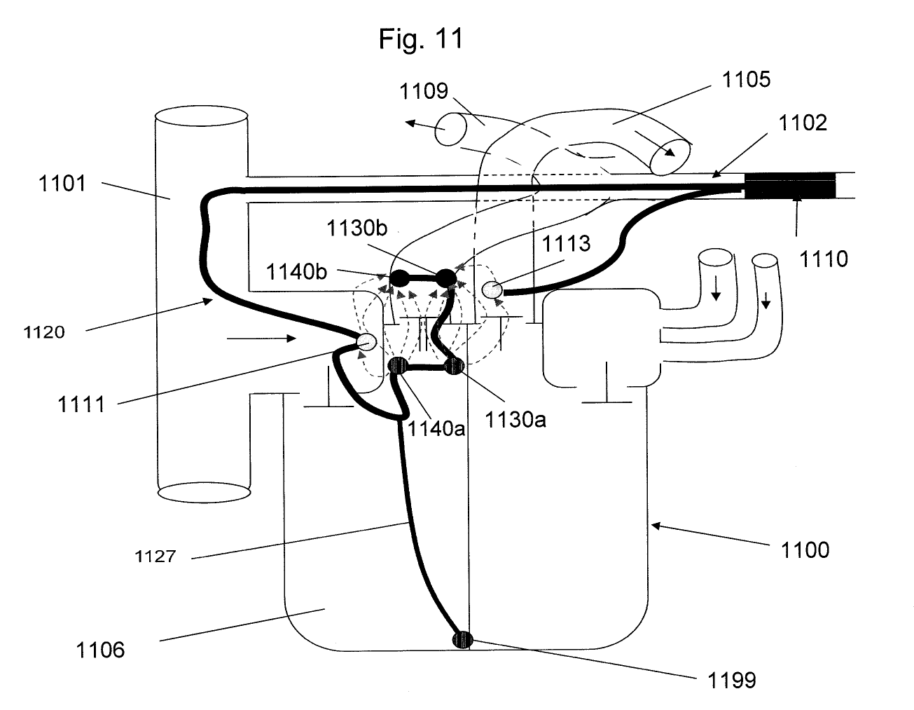

FIG. 11 shows a configuration of electrodes for ventricular defibrillation used for minimizing the magnitude of an electric field outside a defibrillation zone according to some embodiments of the present disclosure. Because some AF patients may also be prone to ventricular fibrillation, it may be advantageous to have both atrial and ventricular defibrillation functionality in one device. For example, FIG. 11 shows the electrode configuration illustrated in FIG. 9E and discussed above, with the addition of a ventricular defibrillation electrode (1199) used for delivering a ventricular defibrillation pulse. In some embodiments, the ventricular defibrillation electrode (1199) may be positioned in the right ventricle (1106) near the cardiac apex. As shown in FIG. 11, the ventricular defibrillation electrode (1199) may be located at the distal end of a secondary single lead (1127) bifurcated from a main single lead (1120). A pulse may be delivered by the ventricular defibrillation electrode (1199) by applying a voltage between the ventricular defibrillation electrode (1199) and the main body (1110) of an IAD according the present disclosure or between the ventricular defibrillation electrode (1199) and any one or several other electrodes, including without limitation electrodes (1130a), (1130b), (1140a) and (1140b) and/or confinement electrodes (1111) and (1113). In some embodiments, the ventricular defibrillation electrode (1199) may be replaced or augmented by other ventricular defibrillation electrodes, including the electrodes (798) and (799) shown in FIG. 7D or as known in the art. In some embodiments, ventricular defibrillation electrodes may be used for ECG sensing, for example, for detecting R-waves and/or for differentiating atrial fibrillation conditions from ventricular fibrillation conditions.

The embodiments set forth in the foregoing description do not represent all embodiments consistent with the subject matter described herein. It is evident that many alternatives, modifications and variations of such embodiments will be apparent to those skilled in the art. As noted elsewhere, these embodiments have been described for illustrative purposes only and are not intended to be limiting. Thus, other embodiments are possible and are covered by the disclosure, which will be apparent from the teachings contained herein. The breadth and scope of the disclosure should not be limited by any of the above-described embodiments but should be defined only in accordance with claims supported by the present disclosure and their equivalents. Moreover, embodiments of the subject disclosure may include methods, systems and devices which may further include any and all elements from any other disclosed methods, systems, and devices; that is, elements from one or another of the disclosed embodiments may be interchangeable with elements from another of the disclosed embodiments. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to any of the disclosed embodiments.

1. An implantable defibrillator comprising:
an electrode lead system having at least one electrode lead and at least three electrodes;
a controller configured to determine whether a heart is fibrillating and emit a command signal if fibrillation exists; and
a voltage generator in communication with the electrode lead system and the controller to sequentially discharge at least a first defibrillation pulse and second defibrillation pulse to the electrode lead system after receiving the command signal,
wherein the first defibrillation pulse creates a first electric field across two of the at least three electrodes and the second defibrillation pulse creates a second electric field across two of the at least three electrodes in a direction different from that of the first electric field.

2. The implantable defibrillator of claim 1, the electrode lead system further comprising a first electrode, a second electrode, a third electrode and a fourth electrode positioned along a single electrode lead,
wherein the first electrode and the second electrode form a first electrode pair and the third electrode and the fourth electrode form a second electrode pair.

3. The implantable defibrillator of claim 2, wherein the first electrode is positioned in the right ventricle, the second electrode and third electrode are positioned in the pulmonary artery and the fourth electrode is positioned in the right atrium.

4. The implantable defibrillator of claim 2, wherein the first electrode is positioned in the right atrium, the second electrode and third electrode are positioned in the pulmonary artery and the fourth electrode is positioned in the right ventricle.

5. The implantable defibrillator of claim 1, the electrode lead system further comprising a first electrode, a second electrode and a third electrode positioned along a single electrode lead,
wherein the first electrode and the second electrode form a first electrode pair and the third electrode and one of the first electrode or the second electrode form a second electrode pair.

6. The implantable defibrillator of claim 5, wherein the first electrode is positioned in the right atrium, the second electrode is positioned in the right ventricle and the third electrode is positioned in the pulmonary artery.

7. The implantable defibrillator of claim 5, wherein the first electrode is positioned in the right atrium, the second electrode is positioned in the pulmonary artery and the third electrode is positioned in the right ventricle.

8. The implantable defibrillator of claim 5, wherein the first electrode is positioned in the pulmonary artery, the second electrode is positioned in the right atrium and the third electrode is positioned in the right ventricle.

9. The implantable defibrillator of claim 5, wherein the first electrode is positioned in the right ventricle, the second electrode is positioned in the right atrium and the third electrode is positioned in the pulmonary artery.

10. The implantable defibrillator of claim 1, the electrode lead system further comprising a first electrode, a second electrode, a third electrode and a fourth electrode positioned along a single electrode lead, wherein:
the first electrode and the fourth electrode receive a negative voltage from the voltage generator and the second electrode and the third electrode receive a positive voltage from the voltage generator to form the first electric field between the first electrode and the second electrode and the first electric field between the third electrode and the fourth electrode; and
the first electrode and the second electrode receive a positive voltage from the voltage generator and the third electrode and the fourth electrode receive a negative voltage from the voltage generator to form the second electric field between the first electrode and the fourth electrode and the second electric field between the second electrode and the third electrode.

11. The implantable defibrillator of claim 10, the first electric fields and the second electric fields intersect each other at different angles to form a high concentration of electrical pulses within a defibrillation zone of the heart.

12. The implantable defibrillator of claim 1, wherein at least one of the at least three electrodes is a confinement electrode configured to control the distribution of at least one of the first electric field or the second electric field.

13. The implantable defibrillator of claim 1, wherein at least one of the at least three electrodes is an extended electrode.

14. The implantable defibrillator of claim 1, wherein at least one of the at least three electrodes is positioned in a ventricle for ventricular defibrillation.

15. The implantable defibrillator of claim 1, wherein the electrode lead system further comprises two single leads.

16. A method for defibrillating the heart with an implantable defibrillator, the method comprising:
positioning an electrode lead system having at least three electrodes in, on and/or around the heart;
determining whether the heart is fibrillating;
if the heart is fibrillating, sending a command signal to a voltage generator;
generating at the voltage generator at least a first defibrillation pulse and second defibrillation pulse;
delivering the first defibrillation pulse and the second defibrillation pulse to the electrode lead system,
wherein the first defibrillation pulse creates a first electric field across two of the at least three electrodes and the second defibrillation pulse creates a second electric field across two of the at least three electrodes in a direction different from that of the first electric field.

17. The method of claim 16, the electrode lead system further comprising a first electrode, a second electrode, a third electrode and a fourth electrode positioned along a single electrode lead,
wherein the first electrode and the second electrode form a first electrode pair and the third electrode and the fourth electrode form a second electrode pair.

18. The method of claim 16, the electrode lead system further comprising a first electrode, a second electrode and a third electrode positioned along a single electrode lead,
wherein the first electrode and the second electrode form a first electrode pair and the third electrode and one of the first electrode or the second electrode form a second electrode pair.

19. The method of claim 16, the electrode lead system further comprising a first electrode, a second electrode, a third electrode and a fourth electrode positioned along a single electrode lead, wherein:
the first electrode and the fourth electrode receive a negative voltage from the voltage generator and the second electrode and the third electrode receive a positive voltage from the voltage generator to form the first electric field between the first electrode and the second electrode and the first electric field between the third electrode and the fourth electrode; and
the first electrode and the second electrode receive a positive voltage from the voltage generator and the third electrode and the fourth electrode receive a negative voltage from the voltage generator to form the second electric field between the first electrode and the fourth electrode and the second electric field between the second electrode and the third electrode.

20. The method of claim 16, wherein at least one of the at least three electrodes is a confinement electrode configured to control the distribution of at least one of the first electric field or the second electric field.

21. The method of claim 16, wherein at least one of the at least three electrodes is an extended electrode.

22. The method of claim 16, wherein at least one of the at least three electrodes is positioned in a ventricle for ventricular defibrillation.

23. The method of claim 16, wherein the electrode lead system further comprises two single leads.

24.-29. (canceled)
